(19) United States
(12) Patent Application Publication
(10) Pub. No.: US 2015/0335021 A1
(43) Pub. Date: Nov. 26, 2015

(54) Cycloclavine and Derivatives Thereof for Controlling Invertebrate Pests

(71) Applicant: BASF SE, Ludwigshafen (DE)

(72) Inventors: Karsten KOERBER, Eppelheim (DE); Dschun SONG, Mannheim (DE); Joachim RHEINHEIMER, Ludwigshafen (DE); Florian KAISER, Mannheim (DE); Joachim DICKHAUT, Heidelberg (DE); Arun NARINE, Mannheim (DE); Deborah L. CULBERTSON, Fuquay Varina, NC (US); Sarah THOMPSON, Raleigh, NC (US); Johann RIEDER, Bad Goegging (DE)

(73) Assignee: BASF SE, Ludwigshafen (DE)

(21) Appl. No.: 14/653,038

(22) PCT Filed: Dec. 19, 2013
(86) PCT No.: PCT/EP2013/077463

§ 371 Date: Jun. 17, 2015

Related U.S. Application Data
(60) Provisional application No. 61/740,461, filed on Dec. 21, 2012.

(51) Int. Cl. A01N 43/90; A01N 47/16; A01N 47/38; C07D 487/06

(52) U.S. Cl. CPC A01N 43/90; C07D 487/06; A01N 47/16; A01N 47/38

(57) ABSTRACT

Indole alkaloid compounds of the general formula (I), salts and N-oxides

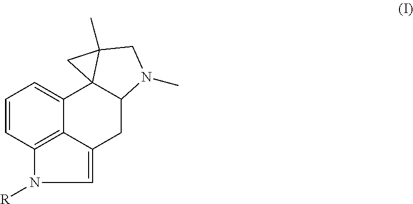

where the symbol in formula (I) has the following meanings given in the specification, are useful for controlling invertebrate pests.

The invention relates to derivatives of cycloclavine, to methods for preparing these compounds and to compositions comprising such compounds. The invention also relates to the use of these compounds, of their salts or of compositions comprising them for controlling invertebrate pests. Furthermore the invention relates to methods of applying such compounds.

Invertebrate pests destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating animal pests.

In particular, invertebrates such as insects and acaridae are difficult to be effectively controlled, and the known compounds are not completely satisfactory in certain cases, in terms, for example, of application rate, spectrum of activity, duration of activity, tendency to form resistance or economic aspects of the preparation process.

It is therefore a continuing objective to provide further pesticidal compounds which, at least in some aspects, offer advantages over the known compounds.

It has been found that particular derivatives of cycloclavine are particularly suitable for controlling pests.

Ergot indole alkaloids in general have been previously described. From this family, cycloclavin, i.e. the compound of formula (I) below where R¹ is H, has been first isolated from Ipomoea Hildebrandtii and characterized by Stauffacher et al, Tetrahedron, (1969), 25, 5879-5887. The results have been verified by Chao et al, Phytochemistry, (1973), 12, 2435-2440. These references provide no biological activity for cycloclavin. Gas chromatography conditions for the identification of cycloclavin have been desribed by Agurell et al, Journal of Chromatography (1971), 61, 339-342. Cycloclavine has been further described by Arens et al, Planta Medica (1980), 39, 336-347. In this reference, radioimmunoassays for the quantitative and separate determination of lysergic acid and simple lysergic acid derivatives have been developed. Lysergic acid was coupled to bovine serum albumin both by the Mannich and the mixed anhydrid reaction and antibodies against these two different conjugates were raised in rabbits, 3H-lysergic acid was synthesized by basic hydrolysis of 3H-ergotamine. The antibodies produced against the conjugate, prepared by the Mannich reaction, were very specific for lysergic acid and did not cross react with any of the other ergot alkaloids tested. The antibodies raised against the conjugate, prepared by the mixed anhydrid reaction, cross reacted with all simple lysergic acid derivatives and some clavines. The antibodies exhibited high affinity towards lysergic acid and its derivatives. However, cycloclavine did not show cross reaction activity in this assay. A method for the isolation of cycloclavin from Aspergillus japonicus has been described by Furuta et al, Agric. Biol. Chem., (1982), 46, 1921-1922. However, no biological activity has been described. Total synthesises of racemic cycloclavin have been described by Incze et al, Tetrahedron (2008), 64, 2924-2929 and Petronijevic et al., JACS 133 (2011) 7704-7707. No significant biological activities of cycloclavine are reported so far. WO 2012/116935 discloses the recombinant manufacture of cycloclavine. Again, no reference to any pesticidal activity is made in this document.

Accordingly, in one aspect of the invention there is provided the use of an indole alkaloid compound of the general formula (I), or a salt or N-oxide thereof for controlling invertebrate pests,

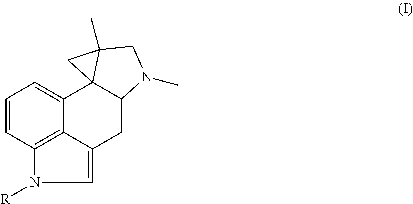

where the symbol in formula (I) has the following meanings:
R is R′, C(═O)R′, C(═O)OR′, C(═O)NR′₂, C(═S)R′, C(═S)OR′, C(═S)NR′₂, C(═NR″)R′, C(═NR″)NR′₂, S(O)ₙR′, S(O)ₙNR′₂, Si(R′″)₃ or NR′₂;
each R′ is hydrogen, C₁-C₆-alkyl, C₂-C₆-alkenyl, C₂-C₆-alkynyl, wherein the carbon atoms of the aforementioned aliphatic radicals are unsubstituted or substituted with one or more R¹, cyano, nitro, nitroso,
   C₃-C₈-cycloalkyl, C₃-C₈-cycloalkenyl, each unsubstituted or substituted with one or more R²,
   phenyl, unsubstituted or substituted with up to five R²,
   a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R″ is independently hydrogen, C₁-C₆ alkyl, C₂-C₆ alkenyl, C₂-C₆ alkynyl, each unsubstituted or substituted with one or more R¹,
   C₃-C₈ cycloalkyl, C₃-C₈ cycloalkenyl, each unsubstituted or substituted with one or more R²,
   phenyl, unsubstituted or substituted with up to five R²,
   a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R′″ is independently C₁-C₆ alkyl, C₁-C₆ haloalkyl, C₁-C₆ alkoxyalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, C₁-C₆ haloalkoxyalkyl,
   phenyl, a 3-, 4-, 5-, -6 or 7-membered saturated, partially unsaturated or aromatic heterocyclic ring containing 1, 2 or 3 heteroatoms or heteroatom groups selected from N, O, S, NO, SO, SO₂;
each R¹ is independently halogen, cyano, azido, nitro, SCN, SF₅ phenyl, unsubstituted or substituted with up to five R²,
   a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
   C═(O)R³, C(═O)OR⁴, C(═O)NR⁵₂, C(═S)R³, C(═S)OR⁴, C(═S)NR⁵₂, C(═NR⁶)R³, C(═NR⁶)NR⁵₂, S(═O)ₙR⁴, S(═O)ₙNR⁵₂, Si(R⁷)₃, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂;
each R² is independently halogen, cyano, azido, nitro, SCN, SF₅,
   C₂-C₆-alkenyl, C₂-C₆-alkynyl, wherein the carbon atoms of the aforementioned aliphatic radicals are unsubstituted or substituted with one or more R^{b},
   C₃-C₈-cycloalkyl, C₃-C₈-cycloalkenyl, each unsubstituted or substituted with one or more R^{a},
   phenyl, unsubstituted or substituted with up to five R^{a},
   a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
   C═(O)R³, C(═O)OR⁴, C(═O)NR⁵₂, C(═S)R³, C(═S)OR⁴, C(═S)NR⁵₂, C(═NR⁶)R³, C(═NR⁶)NR⁵₂, S(═O)ₙR⁴, S(═O)ₙNR⁵₂, Si(R⁷)₃, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, NR⁵—CO—OR⁴;
each R³, is independently hydrogen, cyano, C₁-C₆ alkyl, C₂-C₆ alkenyl, C₂-C₆ alkynyl, wherein the carbon atoms of the aforementioned aliphatic radicals are unsubstituted or substituted with one or more R^{b},
   C₃-C₈ cycloalkyl or C₃-C₈ cycloalkenyl, wherein the carbon atoms of the aforementioned cycloaliphatic radicals are unsubstituted or substituted with one or more R^{a},
   phenyl unsubstituted or substituted with up to 5 R^{a},
   a 3-, 4-, 5-, 6- or 7-membered saturated, partially unsaturated or aromatic heterocyclic ring containing 1, 2 or 3 heteroatoms or heteroatom groups selected from N, O, S, NO, SO, SO₂, wherein the aforementioned ring is unsubstituted or substituted with one or more R^{a};
each R⁴, R⁵ is independently hydrogen, C₁-C₆ alkyl, C₂-C₆ alkenyl, C₂-C₆ alkynyl, wherein the carbon atoms of the aforementioned aliphatic radicals are unsubstituted or substituted with one or more R^{b},
   C₃-C₈ cycloalkyl or C₃-C₈ cycloalkenyl, wherein the carbon atoms of the aforementioned cycloaliphatic radicals are unsubstituted or substituted with one or more R^{a},
   phenyl unsubstituted or substituted with up to 5 R^{a},
   a 3-, 4-, 5-, 6- or 7-membered saturated, partially unsaturated or aromatic heterocyclic ring containing 1, 2 or 3 heteroatoms or heteroatom groups selected from N, O, S, NO, SO, SO₂, wherein the aforementioned ring is unsubstituted or substituted with one or more R^{a};
each R⁶ is independently hydrogen, C₁-C₆ alkyl, C₂-C₆ alkenyl, C₂-C₆ alkynyl, wherein the carbon atoms of the aforementioned aliphatic radicals are unsubstituted or substituted with one or more R^{b},
   C₃-C₈ cycloalkyl or C₃-C₈ cycloalkenyl, wherein the carbon atoms of the aforementioned cycloaliphatic radicals are unsubstituted or substituted with one or more R^{a},
   phenyl unsubstituted or substituted with up to 5 R^{a},
   a 3-, 4-, 5-, 6- or 7-membered saturated, partially unsaturated or aromatic heterocyclic ring containing 1, 2 or 3 heteroatoms or heteroatom groups selected from N, O, S, NO, SO, SO₂, wherein the aforementioned ring is unsubstituted or substituted with one or more R^{a} or OR⁴;
each R⁷ is independently, C₁-C₆ alkyl, C₁-C₆ haloalkyl, C₁-C₆ alkoxyalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, C₁-C₆ haloalkoxyalkyl,
   phenyl, a 3-, 4-, 5-, 6- or 7-membered saturated, partially unsaturated or aromatic heterocyclic ring containing 1, 2 or 3 heteroatoms or heteroatom groups selected from N, O, S, NO, SO, SO₂;
each R^{a} is independently halogen, cyano, azido, nitro, OH, SH, —SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tert-butyldimethylsilyl, C₁-C₆ alkyl, C₁-C₆ haloalkyl, C₂-C₆ n alkenyl, C₂-C₆ haloalkenyl, C₂-C₆ alkynyl, C₂-C₆ haloalkynyl, each unsubstituted or substituted with one or two radicals selected from C₁-C₄ alkoxy and C₁-C₄ haloalkoxy,
   C₃-C₈ cycloalkyl, C₃-C₈ cycloalkenyl, C₃-C₈ halocycloalkyl, C₃-C₈ halocycloalkenyl, each unsubstituted or substituted with one or two radicals selected from C₁-C₄ alkyl and C₁-C₄ alkoxy,
   phenyl, benzyl, pyridyl, phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl;
each R^{b} is independently halogen, cyano, azido, nitro, OH, SH, —SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tert-butyldimethylsilyl,
   C₃-C₈ cycloalkyl, C₃-C₈ cycloalkenyl, C₃-C₈ halocycloalkenyl, C₃-C₈ halocycloalkenyl, each unsubstituted or substituted with one or two radicals selected from C₁-C₄ alkyl and C₁-C₄ alkoxy,
   phenyl, benzyl, pyridyl, phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; and
each n is independently 1 or 2.

In another aspect of the invention there is provided a compound of formula (I) or a salt thereof, with the proviso that R is not H.

In a further aspect of the invention there is provided an agricultural and/or veterinary composition comprising at least one compound of formula (I) or a salt or an N-oxide thereof. In a preferred embodiment said composition further comprises at least one inert liquid and/or at least one solid carrier.

In yet a further aspect of the invention there is provided a method for controlling invertebrate pests, which comprises contacting the invertebrate pests, their habitat, breeding ground, food supply, plant, seed, soil, area, material or environment in which the invertebrate pests are growing or may grow, or the materials, plants, seeds, soils, surfaces or spaces to be protected from animal attack or infestation with a pesticidally effective amount of at least one compound of formula (I) or a salt or an N-oxide thereof.

In a further aspect of the invention there is provided a method for protecting crops from attack or infestation by invertebrate pests, which comprises contacting the crop with a pesticidally effective amount of at least one compound of formula (I) or a salt or an N-oxide thereof.

In yet a further aspect of the invention there is provided a method for protecting seeds from soil insects and the seedlings' roots and shoots from soil and foliar insects, which comprises contacting the seeds before sowing and/or after pregermination with at least one compound of formula (I) or a salt or an N-oxide thereof.

In a further aspect of the invention there are provided seeds comprising at least one compound of formula (I) or a salt or an N-oxide thereof.

In yet a further aspect of the invention there is provided the use of a compound of formula (I) or a salt or an N-oxide thereof for combating parasites in and on animals.

In a further aspect of the invention there is provided a method for treating or protecting animals against infestation or infection by parasites, which comprises orally, topically or parenterally administering or applying to the animals a parasiticidally effective amount of at least one compound of formula (I) or a salt or an N-oxide thereof.

In yet a further aspect of the invention there is provided a method for the preparation of a composition for treating or protecting animals against infestation or infection by parasites, which comprises mixing a parasiticidally effective amount of at least one compound of formula (I) or a salt or an N-oxide thereof and at least one solid carrier.

In a further aspect of the invention there is provided the use of a compound of formula (I) or a salt or an N-oxide thereof for the preparation of a medicament for treating or protecting animals against infestation or infection by parasites.

In yet a further aspect of the invention there is provided a compound of formula (I) or a salt or an N-oxide thereof as a medicament.

In yet a further aspect of the invention there is provided a method for preparing a compound of formula (I), where R H, comprising the step of reacting cycloclavin (I-a)

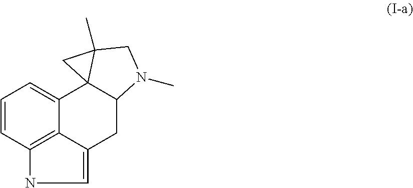 [SMILES: CN1CC2(C)CC23c2cccc4[nH]cc(c24)CC13]

with a compound of formula (II),

R-L (II)

wherein
R is defined as in formula (I) and is ≠H and
L is a leaving group,
optionally in the presence of a base.

The invention also relates to plant propagation materials, in particular seeds, comprising at least one compound of formula (I) or a salt or an N-oxide thereof.

The present invention relates to every possible stereoisomer of the compounds of formula (I), i.e. to single enantiomers or diastereomers, as well as to mixtures thereof.

In particular, formula (I) includes (1aR, 3aR, 9bR)-1a,2,3,3a,4,6-hexahydro-1a,3-dimethyl-1H-cycloprop(c)indolo(4,3-ef)indole, the compound of formula (I-aa):

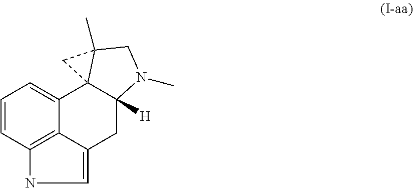

which can be produced in accordance with WO 2012/116935.

The compounds of the invention may be amorphous or may exist in one or more different crystalline states (polymorphs) or modifications which may have different macroscopic properties such as stability or show different biological properties such as activities. The present invention includes both amorphous and crystalline compounds of formula (I), mixtures of different crystalline states or modifications of the respective compound (I), as well as amorphous or crystalline salts thereof.

Salts of the compounds of formula (I) are preferably agriculturally and/or veterinarily acceptable salts. They can be formed in a customary manner, e.g. by reacting the compound with an acid of the anion in question if the compound of formula (I) has a basic functionality or by reacting an acidic compound of formula (I) with a suitable base.

Suitable agriculturally or veterinarilly useful salts are especially the salts of those cations or the acid addition salts of those acids whose cations and anions, respectively, do not have any adverse effect on the action of the compounds according to the present invention.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of formula (I) with an acid of the corresponding anion, preferably of hydrochloric acid, hydrobromic acid, sulfuric acid, phosphoric acid or nitric acid.

Suitable cations are in particular ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

The term “compound of formula (I) or a salt or an N-oxide thereof” includes the compounds, salts thereof, N-oxides thereof and compounds that are both an N-oxide and a salt.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” as used herein refers to fluoro, chloro, bromo and iodo.

The term “Cₙ-Cₘ-alkyl” as used herein (and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl) refers to a branched or unbranched saturated hydrocarbon group having n to m, e.g. 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein (and also in Cₙ-Cₘ-haloalkylsulfinyl and Cₙ-Cₘ-haloalkylsulfonyl) refers to a straight-chain or branched alkyl group having n to m carbon atoms, e.g. 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₆-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, the terms “Cₙ-Cₘ-alkoxy” and “Cₙ-Cₘ-alkylthio” (or the term “Cₙ-Cₘ-alkylsulfenyl”, respectively) refer to straight-chain or branched alkyl groups having n to m carbon atoms, e.g. 1 to 6 or 1 to 4 carbon atoms (as mentioned above) bonded through oxygen or sulfur linkages, respectively, at any bond in the alkyl group. Examples include C₁-C₄-alkoxy such as methoxy, ethoxy, propoxy, isopropoxy, butoxy, sec-butoxy, isobutoxy and tert-butoxy, futher C₁-C₄-alkylthio such as methylthio, ethylthio, propylthio, isopropylthio, and n-butylthio.

Accordingly, the terms “Cₙ-Cₘ-haloalkoxy” and “Cₙ-Cₘ-haloalkylthio” (or the term “Cₙ-Cₘ-haloalkylsulfenyl”, respectively) refer to straight-chain or branched alkyl groups having n to m carbon atoms, e.g. 1 to 6 or 1 to 4 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, ch lorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly the terms “C₁-C₂-fluoroalkoxy” and “C₁-C₂-fluoroalkylthio” refer to C₁-C₂-fluoroalkyl which is bound to the remainder of the molecule via an oxygen atom or a sulfur atom, respectively.

The term “C₂-Cₘ-alkenyl” as used herein refers to a branched or unbranched unsaturated hydrocarbon group having 2 to m, e.g. 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 6 carbon atoms and containing at least one triple bond, such as ethynyl, propynyl, 1-butynyl, 2-butynyl and the like.

The term “C₁-C₄-alkoxy-C₁-C₄-alkyl” as used herein refers to alkyl having 1 to 4 carbon atoms, e.g. like specific examples mentioned above, wherein one hydrogen atom of the alkyl radical is replaced by an C₁-C₄-alkoxy group.

The term “C₃-Cₘ-cycloalkyl” as used herein refers to a monocyclic 3- to m-membered saturated cycloaliphatic radicals, e.g. cyclopropyl, cyclobutyl, cyclopentyl, cyclohexyl, cycloheptyl and cyclooctyl.

The term “3-, 4-, 5-, 6- or 7-membered saturated, partially unsaturated or aromatic heterocyclic ring containing 1, 2 or 3 heteroatoms or heteroatom groups selected from N, O, S, NO, SO, SO₂” as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclyl include: oxiranyl, aziridinyl, azetidinyl, 2 tetrahydrofuranyl, 3-tetrahydrofuranyl, 2 tetrahydrothienyl, 3 tetrahydrothienyl, 2-pyrrolidinyl, 3-pyrrolidinyl, 3 pyrazolidinyl, 4 pyrazolidinyl, 5-pyrazolidinyl, 2 imidazolidinyl, 4 imidazolidinyl, 2-oxazolidinyl, 4-oxazolidinyl, 5 oxazolidinyl, 3-isoxazolidinyl, 4 isoxazolidinyl, 5 isoxazolidinyl, 2 thiazolidinyl, 4-thiazolidinyl, 5-thiazolidinyl, 3 isothiazolidinyl, 4-isothiazolidinyl, 5 isothiazolidinyl, 1,2,4-oxadiazolidin-3-yl, 1,2,4 oxadiazolidin 5 yl, 1,2,4-thiadiazolidin-3-yl, 1,2,4 thiadiazolidin-5-yl, 1,2,4 triazolidin-3-yl, 1,3,4-oxadiazolidin-2-yl, 1,3,4 thiadiazolidin-2-yl, 1,3,4 triazolidin-2-yl, 2-tetrahydropyranyl, 4 tetrahydropyranyl, 1,3-dioxan-5-yl, 1,4-dioxan-2-yl, 2-piperidinyl, 3-piperidinyl, 4-piperidinyl, 3-hexahydropyridazinyl, 4 hexahydropyridazinyl, 2-hexahydropyrimidinyl, 4-hexahydropyrimidinyl, 5 hexahydropyrimidinyl, 2-piperazinyl, 1,3,5-hexahydrotriazin-2-yl and 1,2,4 hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3 dihydrothien-3-yl, 2,4 dihydrothien-2-yl, 2,4-dihydrothien-3-yl, 2-pyrrolin-2-yl, 2-pyrrolin-3-yl, 3 pyrrolin-2-yl, 3-pyrrolin-3-yl, 2-isoxazolin-3-yl, 3-isoxazolin-3-yl, 4 isoxazolin 3 yl, 2-isoxazolin-4-yl, 3-isoxazolin-4-yl, 4-isoxazolin-4-yl, 2 isoxazolin-5-yl, 3-isoxazolin-5-yl, 4-isoxazolin-5-yl, 2-isothiazolin-3-yl, 3 isothiazolin-3-yl, 4-isothiazolin-3-yl, 2-isothiazolin-4-yl, 3-isothiazolin-4-yl, 4 isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3 dihydropyrazol-4-yl, 2,3-dihydropyrazol-5-yl, 3,4-dihydropyrazol-1-yl, 3,4 dihydropyrazol-3-yl, 3,4-dihydropyrazol-4-yl, 3,4-dihydropyrazol-5-yl, 4,5 dihydropyrazol-1-yl, 4,5-dihydropyrazol-3-yl, 4,5-dihydropyrazol-4-yl, 4,5 dihydropyrazol-5-yl, 2,3-dihydrooxazol-2-yl, 2,3-dihydrooxazol-3-yl, 2,3 dihydrooxazol-4-yl, 2,3-dihydrooxazol-5-yl, 3,4-dihydrooxazol-2-yl, 3,4 dihydrooxazol-3-yl, 3,4-dihydrooxazol-4-yl, 3,4-dihydrooxazol-5-yl, 3,4 dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4 di- or tetrahydropyridazinyl, 2-di- or tetrahydropyrimidinyl, 4-di- or tetrahydropyrimidinyl, 5 di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7 tetrahydro[1H]azepin-1-, -2-, -3-, -4-, -5-, -6- or -7-yl, 2,3,6,7 tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7 tetrahydro[1 H]oxepin-2-, -3-, -4-, -5-, -6- or -7-yl, 2,3,6,7 tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

3-, 4-, 5-, 6- or 7-membered aromatic heterocyclyl is 5- or 6-membered aromatic heterocyclyl (hetaryl). Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4 thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

Preferably, the term “phenyl unsubstituted or substituted with 1, 2, 3, 4 or 5 substituents R²/R^{a} means “phenyl unsubstituted or substituted with up to 3 or in the case of halogen up to the maximum possible number of substituents R²/R^{a}, and also preferably “phenyl unsubstituted or substituted with 1, 2, 3 or 4 substituents R²/R^{a}, more preferably “phenyl unsubstituted or substituted with 1, 2 or 3 substituents R²/R^{a}, even more preferably “phenyl unsubstituted or substituted with 1 or 2 substituents R²/R^{a}, and particularly preferably “phenyl unsubstituted or substituted with 1 substituent R²/R^{a}.

Preferably, the term “unsubstituted or substituted with one or more”, in connection with substituents R¹, R², R^{a}, or R^{b}, means “unsubstituted or substituted with up to 5 or in the case of halogen up to the maximum possible number of”, more preferably “unsubstituted or substituted with up to 3 or in the case of halogen up to the maximum possible number of”, even more preferably “unsubstituted or substituted with up to 2 or in the case of halogen up to the maximum possible number of”, also more preferably “unsubstituted or substituted with up to 5”, also even more preferably “unsubstituted or substituted with up to 3”, and particularly preferably “unsubstituted or substituted with up to 2”.

The preferred, more preferred, even more preferred and particularly preferred substituents and embodiments described herein are to be understood as preferred either independently of each other or in every possible combination with each other.

These preferences and embodiments apply to the compounds of the invention, to the use of the compounds of the invention as well as to methods using the compounds of the invention.

In preferred embodiments of the invention the symbols in formula (I), each independently, have the following meanings.
R is preferably R′, C(═O)R′, C(═O)OR′, C(═O)NR′₂, C(═S)R′, C(═S)OR′, C(═S)NR′₂, C(═NR″)R′, C(═NR″)NR′₂, S(O)ₙR′, S(O)nNR′₂ or NR′₂.
Each R′ is preferably independently hydrogen, C₁-C₆-alkyl, C₂-C₆-alkenyl, wherein the carbon atoms of the aforementioned aliphatic radicals are unsubstituted or substituted with one or more R¹, cyano,
   C₃-C₈-cycloalkyl, C₅-C₆-cycloalkenyl, each unsubstituted or substituted with one or more R²,
   phenyl, unsubstituted or substituted with up to five R²,
   a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R².
Each R″ is preferably independently hydrogen, C₁-C₆ alkyl, C₂-C₆ alkenyl, each unsubstituted or substituted with one or more R¹,
C₃-C₈ cycloalkyl, C₅-C₆ cycloalkenyl, each unsubstituted or substituted with one or more R²,
   phenyl, unsubstituted or substituted with up to five R²,
   a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R².
Each R′″ is preferably independently C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl or
   phenyl.
Each R¹ is preferably independently halogen, cyano,
   phenyl, unsubstituted or substituted with up to five R²,
   a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
   C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂.
Each R² is preferably independently halogen, cyano,
   C₁-C₆-alkyl, C₂-C₆-alkenyl, wherein the carbon atoms of the aforementioned aliphatic radicals are unsubstituted or substituted with one or more Rb,
   C₃-C₈-cycloalkyl, C₅-C₆-cycloalkenyl, each unsubstituted or substituted with one or more R^{a},
   phenyl, unsubstituted or substituted with up to five R^{a},
   a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
   C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, NR⁵—CO—OR⁴.
Each R³, R⁴, R⁵ is preferably independently hydrogen, C₁-C₆ alkyl, wherein the carbon atoms of the aforementioned aliphatic radicals are unsubstituted or substituted with one or more R^{b},
   C₃-C₈ cycloalkyl or C₅-C₆ cycloalkenyl, wherein the carbon atoms of the aforementioned cycloaliphatic radicals are unsubstituted or substituted with one or more Ra,
   phenyl unsubstituted or substituted with up to 5 R^{a},
   a 3-, 4-, 5- or 6-membered saturated, partially unsaturated or aromatic heterocyclic ring containing 1, 2 or 3 heteroatoms or heteroatom groups selected from N, O, S, NO, SO, SO₂, wherein the aforementioned ring is unsubstituted or substituted with one or more Ra.
Each R⁶ is preferably independently hydrogen, C₁-C₆ alkyl, C₂-C₆ alkenyl, wherein the carbon atoms of the aforementioned aliphatic radicals are unsubstituted or substituted with one or more R^{b},
   C₃-C₈ cycloalkyl or C₅-C₆ cycloalkenyl, wherein the carbon atoms of the aforementioned cycloaliphatic radicals are unsubstituted or substituted with one or more R^{a},
   phenyl unsubstituted or substituted with up to 5 R^{a},
   a 3-, 4-, 5- or 6-membered saturated, partially unsaturated or aromatic heterocyclic ring containing 1, 2 or 3 heteroatoms or heteroatom groups selected from N, O, S, NO, SO, SO₂, wherein the aforementioned ring is unsubstituted or substituted with one or more R^{a} or OR⁴.
Each R⁷ is preferably independently, C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl,
   phenyl, a 3-, 4-, 5- or 6-membered saturated, partially unsaturated or aromatic heterocyclic ring containing 1, 2 or 3 heteroatoms or heteroatom groups selected from N, O, S, NO, SO, SO₂.
Each R^{a} is preferably independently halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆ alkyl, C₁-C₆ haloalkyl, C₂-C₆ alkenyl, C₂-C₆ haloalkenyl, each unsubstituted or substituted with one or two radicals selected from C₁-C₄ alkoxy and C₁-C₄ haloalkoxy,
   C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
   phenyl, benzyl, pyridyl, phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl.
Each R^{b} is preferably independently halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio,
   C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
   phenyl, benzyl, pyridyl, phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl.
Each n is preferably independently 1 or 2.

Further preferred are compounds of formula (I), salts and N-oxides thereof, where all symbols have the preferred meanings.

In more preferred embodiments of the invention the symbols in formula (I), each independently, have the following meanings.
R is more preferred R′, NR′₂, C(═O)R′, C(═O)OR′, C(═O)NR′₂.
Each R′ is more preferred independently hydrogen,
   C₁-C₆-alkyl, C₂-C₆-alkenyl, each substituted with one or more R¹,
   phenyl, substituted with one or two R²,
   or is selected from:

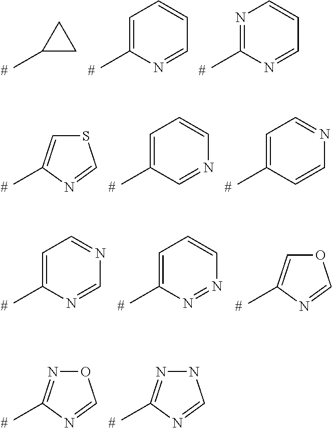

wherein each of the above ring systems is unsubstituted or substituted with one or more, preferably one or two, R².
Each R¹ is more preferred independently halogen.
Each R² is more preferred independently halogen, C₁-C₆ alkyl, C₁-C₆ haloalkyl or OR⁴.
Each R⁴ is more preferred independently C₁-C₆ alkyl or C₁-C₆ haloalkyl.

More preferred are further compounds of formula (I) and salts thereof, where all symbols have the more preferred meanings.

In particularly preferred embodiments of the invention the symbols in formula (I), each independently, have the following meanings.
R is particularly preferred R′, NR′₂, C(═O)R′, C(═O)OR′, C(═O)NR′₂.
Each R′ is particularly preferred hydrogen,
   C₁-C₆-alkyl, C₂-C₆-alkenyl, each substituted with one or more R¹,
   phenyl, substituted with one or two R²,
   or is selected from A1 to A28:

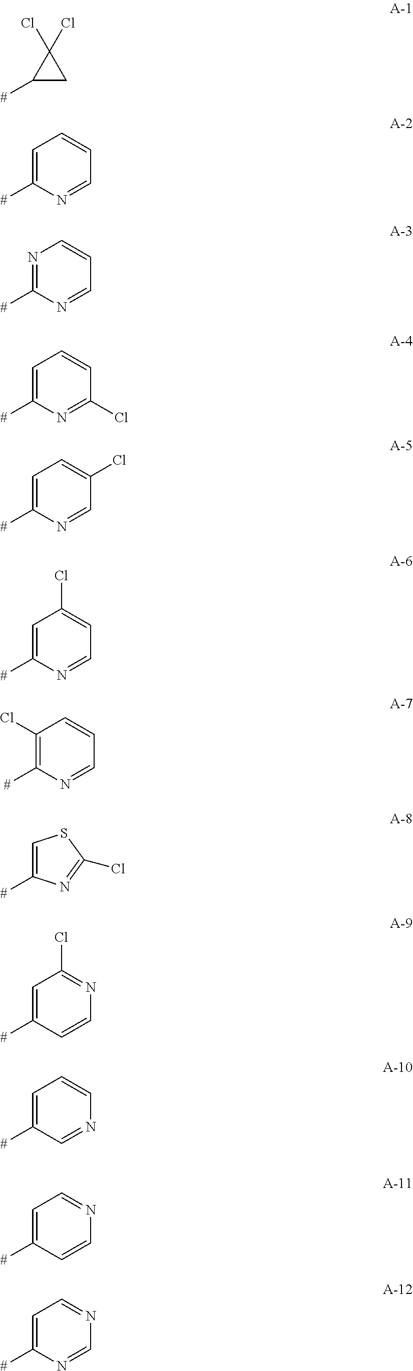

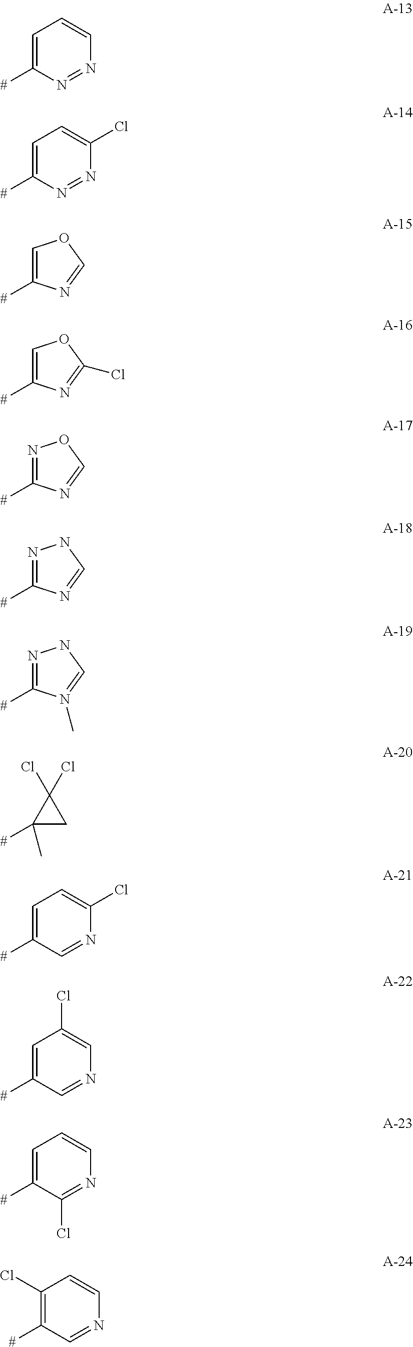

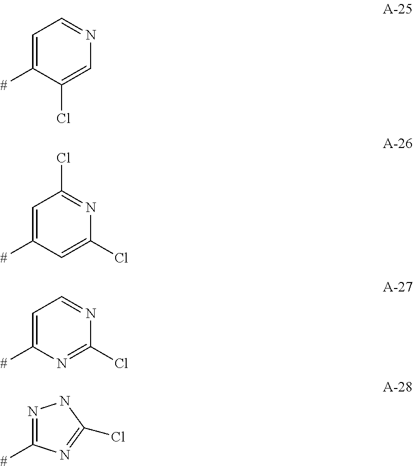

Each R¹ is particularly preferred independently halogen.
Each R² is particularly preferred independently halogen, C₁-C₆ alkyl, C₁-C₆ haloalkyl or OR⁴.
Each R⁴ is particularly preferred independently C₁-C₆ alkyl or C₁-C₆ haloalkyl.

Further particularly preferred are compounds of formula (I), salts and N-oxdies thereof, where all symbols have the particularly preferred meanings.
Further preferred are the compounds of formula (I), listed in Table A below,

wherein the variables A1-A28 have the meanings given above and

wherein “#” indicates the bond to nitrogen in formula (I).

TABLE A

| No. | R |
|---|---|
| 1 | H |
| 2 | CH₃ |
| 3 | NHCH₃ |
| 4 | NHCH₂CH₃ |
| 5 | NHCH₂CH₂CH₃ |
| 6 | NHCH₂CF₃ |
| 7 | CH₂CF₃ |
| 8 | CH₂CH₃ |
| 9 | CH₂CH₂CH₃ |
| 10 | CH₂CH₂CH₂CH₃ |
| 11 | CF₃ |
| 12 | CH(CH₃)₂ |
| 13 | C(═O)—CH₃ |
| 14 | C(═O)—CH₂CH₃ |
| 15 | C(═O)—CH₂CH₂CH₃ |
| 16 | C(═O)—CH(CH₃)₂ |
| 17 | C(═O)—CH₂CH₂CH₂CH₃ |
| 18 | C(═O)—CF₃ |
| 19 | C(═O)—CH₂CF₃ |
| 20 | C(═O)O—CH₃ |
| 21 | C(═O)O—CH₂CH₃ |
| 22 | C(═O)O—CH₂CH₂CH₃ |
| 23 | C(═O)O—CH(CH₃)₂ |
| 24 | C(═O)O—CH₂C₆H₅ |
| 25 | C(═O)NH—CH₃ |
| 26 | C(═O)NH—CH₂CH₂CH₃ |
| 27 | C(═O)NH—CH₂C₆H₅ |
| 28 | C(═O)N(CH₃)₂ |
| 29 | Ph |
| 30 | CH₂Ph |
| 31 | CH₂CH═CH-4-Cl—C₆H₄ |
| 32 | CH₂CH═CH-4-CF₃—C₆H₄ |
| 33 | 2-Cl—C₆H₄ |
| 34 | 3-Cl—C₆H₄ |
| 35 | 4-Cl—C₆H₄ |
| 36 | 2-CH₃—C₆H₄ |
| 37 | 3-CH₃—C₆H₄ |
| 38 | 4-CF₃—C₆H₄ |
| 39 | 2-CF₃—C₆H₄ |
| 40 | 3-CF₃—C₆H₄ |
| 41 | 4-CH₃—C₆H₄ |
| 42 | 2-OCH₃—C₆H₄ |
| 43 | 3-OCH₃—C₆H₄ |
| 44 | 4-OCH₃—C₆H₄ |
| 45 | 2-OCF₃—C₆H₄ |
| 46 | 3-OCF₃—C₆H₄ |
| 47 | 4-OCF₃—C₆H₄ |
| 48 | A-2 |
| 49 | A-3 |
| 50 | A-4 |
| 51 | A-5 |
| 52 | A-6 |
| 53 | A-7 |
| 54 | A-8 |
| 55 | A-9 |
| 56 | A-10 |
| 57 | A-11 |
| 58 | A-12 |
| 59 | A-13 |
| 60 | A-14 |
| 61 | A-15 |
| 62 | A-16 |
| 63 | A-17 |
| 64 | A-18 |
| 65 | A-19 |
| 66 | A-21 |
| 67 | A-22 |
| 68 | A-23 |
| 69 | A-24 |
| 70 | A-25 |
| 71 | A-26 |
| 72 | A-27 |
| 73 | A-28 |
| 74 | C(═O)—Ph |
| 75 | C(═O)—CH₂Ph |
| 76 | C(═O)-2-Cl—C₆H₄ |
| 77 | C(═O)-3-Cl—C₆H₄ |
| 78 | C(═O)-4-Cl—C₆H₄ |
| 79 | C(═O)-2-CH₃—C₆H₄ |
| 80 | C(═O)-3-CH₃—C₆H₄ |
| 81 | C(═O)-4-CF₃—C₆H₄ |
| 82 | C(═O)-2-CF₃—C₆H₄ |
| 83 | C(═O)-3-CF₃—C₆H₄ |
| 84 | C(═O)-4-CH₃—C₆H₄ |
| 85 | C(═O)-2-OCH₃—C₆H₄ |
| 86 | C(═O)-3-OCH₃—C₆H₄ |
| 87 | C(═O)-4-OCH₃—C₆H₄ |
| 88 | C(═O)-2-OCF₃—C₆H₄ |
| 89 | C(═O)-3-OCF₃—C₆H₄ |
| 90 | C(═O)-4-OCF₃—C₆H₄ |
| 91 | C(═O)-A-2 |
| 92 | C(═O)-A-3 |
| 93 | C(═O)-A-4 |
| 94 | C(═O)-A-5 |
| 95 | C(═O)-A-6 |
| 96 | C(═O)-A-7 |
| 97 | C(═O)-A-8 |
| 98 | C(═O)-A-9 |
| 99 | C(═O)-A-10 |
| 100 | C(═O)-A-11 |
| 101 | C(═O)-A-12 |
| 102 | C(═O)-A-13 |
| 103 | C(═O)-A-14 |
| 104 | C(═O)-A-15 |
| 105 | C(═O)-A-16 |
| 106 | C(═O)-A-17 |
| 107 | C(═O)-A-18 |
| 108 | C(═O)-A-19 |
| 109 | C(═O)-A-21 |
| 110 | C(═O)-A-22 |
| 111 | C(═O)-A-23 |
| 112 | C(═O)-A-24 |
| 113 | C(═O)-A-25 |
| 114 | C(═O)-A-26 |
| 115 | C(═O)-A-27 |
| 116 | C(═O)-A-28 |

### Preparation Methods

Compounds of formula (I) can be prepared e.g. according the preparation methods and preparation schemes as described below.

Methods for the preparation of substituted indole alkaloid compounds of formula (I): Cycloclavin of formula (I-a) may be obtained according to one of the published methods by Stauffacher et al, Tetrahedron, (1969), 25, 5879-5887 or Chao et al, Phytochemistry, (1973), 12, 2435-2440. Alternatively, cycloclavine may be yielded from fermentation methods, as for example described by Furuta et al, Agric. Biol. Chem., (1982), 46,1921-1922. In a preferred embodiment cycloclavin, in particular the compound of formula (I-aa), is prepared by the method of WO 2012/116935, the content of which is hereby incorporated by reference.

Alternatively, racemic cycloclavin may be synthetically prepared as for example described by Incze et al, Tetrahedron (2008), 64, 2924-2929 or F. R. Petronijevic et al., JACS 113 (2011) 7704-7707. In general, compounds of formula (I) can be prepared by a method, comprising the step of reacting the compound of formula (Ia)

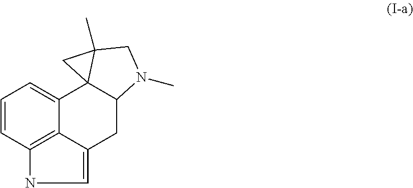 [SMILES: CN1CC2(C)CC23c2cccc4[nH]cc(c24)CC13]

with a compound of formula (II),

R-L (II)

wherein
R is defined as in formula (I) and is ≠H and
L is a leaving group, e.g. halogen, mesylate, trifluoromesylate or tosylate, optionally in the presence of a base.

Preferably, compounds of formula (I), where R≠H, can be prepared from cycloclavin according to the following methods and variations described in schemes 1-3.

Compounds of formula I-b can, for example, be prepared as shown in Scheme 1 from cycloclavin (I-a) by reaction with an acid chloride or an acid anhydride, as for example described by Capelli et al, Journal of Organic Chemistry (2009), 74, 7191-7194 (G in this case is a radical R′).

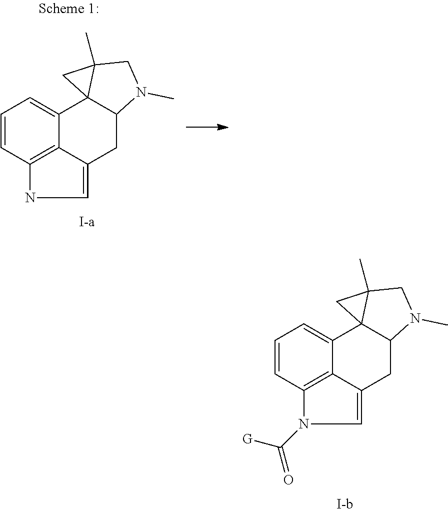

Alternatively, compounds of formula I-b can be prepared by the reaction of cycloclavin with an isocyanate, as for example described in WO 2008/048981 (G in this case is a radical NR′₂).

Compounds of formula I-c can be prepared as shown in Scheme 2 by alkylation of cycloclavin as for example described by Muro et al, Journal of Medicinal Chemistry (2009), 52, 7974-7992. (A in this case is a radical selected independently from C₁-C₆-alkyl, C₁-C₆-haloalkyl, C₁-C₆-alkylsulfinyl, C₁-C₆-alkylsulfonyl, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, wherein the carbon atoms of the aforementioned aliphatic and cyclo-aliphatic radicals may optionally be substituted with one or more R¹, R².

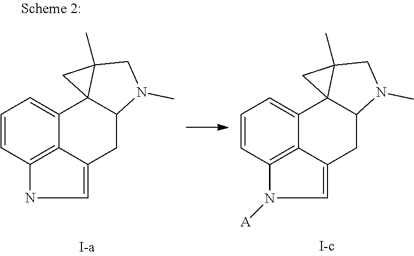

Alternatively, compounds of formula I-c can be prepared by a transition metal catalyzed aryl coupling, as for example described by Mutule et al, Journal of Organic Chemistry (2009), 74, 7195-7198. (A in this case is a radical selected independently from phenyl, optionally substituted with one or more substituents R⁵, which are independently selected from one another, a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with k substituents R⁵, selected independently from one another, and wherein the nitrogen and/or the sulfur atom(s) of the heterocyclic ring may optionally be oxidized).

Compounds of formula I-d can also be prepared as shown in Scheme 3 by reaction of cyclicloclavin with an azanyl ester, as for example described in US 2008/292626 (J in this case may be a radical NR′₂). Alternatively, compounds of formula I-d can also be prepared from cycloclavin by reaction of cycloclavin with a nitration reagent or a nitrosation reagent as for example described by Liebeskind et al, Organic & Biomolecular Chemistry (2008), 6, 2560-2573 or Kyziol et al., Liebigs Annalen der Chemie (1985), 1336-1345 (J in this case is nitro or nitroso).

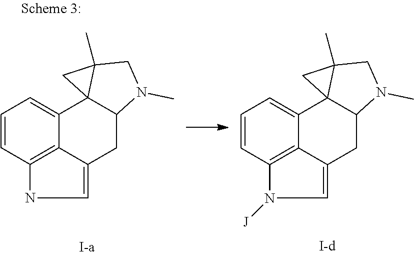

N-oxides of the compounds of formula I can be formed in a customary manner, e.g. by treating a compound of formula I with a suitable oxidant. Examples of suitable oxidants include hydrogen peroxide, urea hydrogen peroxide (UHP), meta-chloroperbenzoic acid (mCPBA), sodium perborate, sodium percarbonate.

If individual compounds cannot be prepared via the above-described routes, they can be prepared by derivatization of other compounds (I) or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization or digestion.

### Pests

The term “invertebrate pest” as used herein encompasses animal populations, such as arthropode pests, including insects and arachnids, as well as nematodes, which may attack plants thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The compounds of formula (I) and their salts are in particular suitable for efficiently controlling arthropodal pests such as arachnids, myriapedes and insects as well as nematodes.

The compounds of formula (I) are especially suitable for efficiently combating the following pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera l ssp., Diabrotica longicornis, Diabrotica semipunctata, Diabrotica 12-punctata Diabrotica speciosa, Diabrotica virgifera, Epllachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleanae, Phyllobius gyri, Phyllotreta chrysocephala, Phyllophaga sp., Phyllopertha horticola, Phyllotreta nemorum, Phyllotreta striolata, Popillia japonica, Sitona kneatus and Sitophilus granaria;

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprin, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;

cockroaches (Blattaria-Blattodea), e.g. Blattella germanica, Blattella asahinae, Periplanta americana, Periplaneta japonica, Periplaneta brunnea, Periplaneta fuligginosa, Periplaneta australasiae, and Blatta orientalis;

bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara vindula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolli, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharcida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus;

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;

arachnids (Arachnoidea), such as acarians (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni, Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;

fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharin and Thermobia domestica,
centipedes (Chilopoda), e.g. Scutigera coleoptrata,
millipedes (Diplopoda), e.g. Narceus spp.,
Earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
Collembola (springtails), e.g. Onychiurus ssp.

They are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolairnus longicaudatus and other Belonolaimus species; Pine nematodes, Bursaphelenchus xylophllus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

Compounds of the formula I are particularly useful for controlling insects, preferably sucking or piercing insects such as insects from the genera Thysanoptera, Diptera and Hemiptera, in particular the following species:

Thysanoptera: Frankliniella fusca, Frankliniella occidentalis, Frankliniella tritici, Scirtothrips citri, Thrips oryzae, Thrips palmi and Thrips tabaci,

Diptera, e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliompa hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucllia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

Hemiptera, in particular aphids: Acyrthosiphon onobrychis, Adelges laricis, Aphidula nasturtii, Aphis fabae, Aphis forbesi, Aphis pomi, Aphis gossypi, Aphis grossulanae, Aphis schneideri, Aphis spiraecola, Aphis sambuci, Acyrthosiphon Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis gyri, Empoasca fabae, Hyalopterus Pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Tnaleurodes vaporariorum, Toxoptera aurantiiand, and Viteus vitifolii.

Compounds of formula (I) are particularly useful for controlling insects of the orders Hemiptera and Thysanoptera.

### Formulations

The invention also relates to agrochemical compositions comprising an auxiliary and at least one compound of formula (I).

An agrochemical composition comprises a pesticidally effective amount of a compound (I). The term “effective amount” denotes an amount of the composition or of the compounds (I), which is sufficient for controlling harmful pests on cultivated plants or in the protection of materials and which does not result in a substantial damage to the treated plants. Such an amount can vary in a broad range and is dependent on various factors, such as the animal pests species to be controlled, the treated cultivated plant or material, the climatic conditions and the specific compound (I) used.

The compounds (I) and their salts can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Mono-graph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, lime-stone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and am-photeric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsul-fonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanofer-rate) and organic colorants (e.g. alizarin-, azo- and phthalocyanine colorants).

Suitable tackifiers or binders are polyvinylpyrrolidons, polyvinylacetates, polyvinyl alcohols, pol-yacrylates, biological or synthetic waxes, and cellulose ethers.

Examples for composition types and their preparation are:
i) Water-soluble concentrates (SL, LS)

10-60 wt % of a compound I according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.
ii) Dispersible concentrates (DC)

5-25 wt % of a compound I according to the invention and 1-10 wt % dispersant (e.g. polyvinylpyrrolidone) are dissolved in organic solvent (e.g. cyclohexanone) ad 100 wt %. Dilution with water gives a dispersion.
iii) Emulsifiable concentrates (EC)

15-70 wt % of a compound I according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.
iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.
v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.
vi) Water-dispersible granules and water-soluble granules (WG, SG)

50-80 wt % of a compound I according to the invention are ground finely with addition of dispersants and wetting agents (e.g. sodium lignosulfonate and alcohol ethoxylate) ad 100 wt %and prepared as water-dispersible or water-soluble granules by means of technical appliances (e.g. extrusion, spray tower, fluidized bed). Dilution with water gives a stable dispersion or solution of the active substance.
vii) Water-dispersible powders and water-soluble powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.
viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.
iv) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water ad 100%. This mixture is stirred for 1 h to produce spontaneously a thermodynamically stable microemulsion.
iv) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solu-tion of a protective colloid (e.g. polyvinyl alcohol). The addition of a polyamine (e.g. hexa-methylenediamine) results in the formation of a polyurea microcapsules. The monomers amount to 1-10 wt %. The wt % relate to the total CS composition.
ix) Dustable powders (DP, DS)

1-10 wt % of a compound I according to the invention are ground finely and mixed intimately with solid carrier (e.g. finely divided kaolin) ad 100 wt %.
x) Granules (GR, FG)

0.5-30 wt % of a compound I according to the invention is ground finely and associated with solid carrier (e.g. silicate) ad 100 wt %. Granulation is achieved by extrusion, spray-drying or the fluidized bed.
xi) Ultra-low volume liquids (UL)

1-50 wt % of a compound I according to the invention are dissolved in organic solvent (e.g. aromatic hydrocarbon) ad 100 wt %.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (ac-cording to NMR spectrum).

Solutions for seed treatment (LS), Suspo-emulsions (SE), flowable concentrates (FS), powders for dry treatment (DS), water-dispersible powders for slurry treatment (WS), water-soluble pow-ders (SS), emulsions (ES), emulsifiable concentrates (EC) and gels (GF) are usually employed for the purposes of treatment of plant propagation materials, particularly seeds. The composi-tions in question give, after two-to-tenfold dilution, active substance concentrations of from 0.01 to 60% by weight, preferably from 0.1 to 40% by weight, in the ready-to-use preparations. Appli-cation can be carried out before or during sowing. Methods for applying compound I and com-positions thereof, respectively, on to plant propagation material, especially seeds include dress-ing, coating, pelleting, dusting, soaking and in-furrow application methods of the propagation material. Preferably, compound I or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha. In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemi-cal composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the in-vention such as parts of a kit or parts of a binary or ternary mixture may be mixed by the user himself in a spray tank and further auxiliaries may be added, if appropriate.

In a further embodiment, either individual components of the composition according to the in-vention or partially premixed components, e.g. components comprising compounds I, may be mixed by the user in a spray tank and further auxiliaries and additives may be added, if appropriate.

In a further embodiment, either individual components of the composition according to the invention or partially premixed components, e.g. components comprising compounds I, can be applied jointly (e.g. after tank mix) or consecutively.

### Mixtures

According to one embodiment of the present invention, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e.g. components comprising compounds I and/or active substances from the groups M.1 to M.UN.X or F.I to F.XII, may be mixed by the user in a spray tank and fur-ther auxiliaries and additives may be added, if appropriate.

In a further embodiment, either individual components of the composition according to the invention or partially premixed components, e.g. components comprising compounds I and/or active substances from the groups M.1 to M.UN.X or F.I to F.XII, can be applied jointly (e.g. after tank mix) or consecutively.

The following list M of pesticides, grouped according the Mode of Action Classification of the Insecticide Resistance Action Committee (IRAC), together with which the compounds according to the invention can be used and with which potential synergistic effects might be produced, is intended to illustrate the possible combinations, but not to impose any limitation:
M.1 Acetylcholine esterase (AChE) inhibitors from the class of
M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathionmethyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
M.2. GABA-gated chloride channel antagonists such as:
M.2A cyclodiene organochlorine compounds, as for example endosulfan or chlordane; or
M.2B fiproles (phenylpyrazoles), as for example ethiprole, fipronil, flufiprole, pyrafluprole and pyriprole;
M.3 Sodium channel modulators from the class of
M.3A pyrethroids, for example acrinathrin, allethrin, d-cis-trans allethrin, d-trans allethrin, bifenthrin, bioallethrin, bioallethrin 5-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin,metofluthrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
M.3B sodium channel modulators such as DDT or methoxychlor;
M.4 Nicotinic acetylcholine receptor agonists (nAChR) from the class of
M.4A neonicotinoids, for example acteamiprid, chlothianidin, dinotefuran, imidacloprid, nitenpyram, thiacloprid and thiamethoxam; or M.4B nicotine.
M.5 Nicotinic acetylcholine receptor allosteric activators from the class of spinosyns, for example spinosad or spinetoram;
M.6 Chloride channel activators from the class of avermectins and milbemycins, for example abamectin, emamectin benzoate, ivermectin, lepimectin or milbemectin;
M.7 Juvenile hormone mimics, such as
M.7A juvenile hormone analogues as hydroprene, kinoprene and methoprene; or others as
M.7B fenoxycarb or M.7C pyriproxyfen;
M.8 miscellaneous non-specific (multi-site) inhibitors, for example
M.8A alkyl halides as methyl bromide and other alkyl halides, or
M.8B chloropicrin, or M.8C sulfuryl fluoride, or M.8D borax, or M.8E tartar emetic;
M.9 Selective homopteran feeding blockers, for example
M.9B pymetrozine, or M.9C flonicamid;
M.10 Mite growth inhibitors, for example
M.10A clofentezine, hexythiazox and diflovidazin, or M.10B etoxazole;
M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;
M.12 Inhibitors of mitochondrial ATP synthase, for example
M.12A diafenthiuron, or
M.12B organotin miticides such as azocyclotin, cyhexatin or fenbutatin oxide, or M.12C propargite, or M.12D tetradifon;
M.13 Uncouplers of oxidative phosphorylation via disruption of the proton gradient, for example chlorfenapyr, DNOC or sulfluramid;
M.14 Nicotinic acetylcholine receptor (nAChR) channel blockers, for example nereistoxin analogues as bensultap, cartap hydrochloride, thiocyclam or thiosultap sodium;
M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;
M.16 Inhibitors of the chitin biosynthesis type 1, as for example buprofezin;
M.17 Moulting disruptors, Dipteran, as for example cyromazine;
M.18 Ecdyson receptor agonists such as diacylhydrazines, for example methoxyfenozide, tebufenozide, halofenozide, fufenozide or chromafenozide;
M.19 Octopamin receptor agonists, as for example amitraz;
M.20 Mitochondrial complex III electron transport inhibitors, for example
M.20A hydramethylnon, or M.20B acequinocyl, or M.20C fluacrypyrim;
M.21 Mitochondrial complex I electron transport inhibitors, for example
M.21A METI acaricides and insecticides such as fenazaquin, fenpyroximate, pyrimidifen, pyridaben, tebufenpyrad or tolfenpyrad, or M.21 B rotenone;
M.22 Voltage-dependent sodium channel blockers, for example
M.22A indoxacarb, or M.22B metaflumizone;
M.23 Inhibitors of the of acetyl CoA carboxylase, such as Tetronic and Tetramic acid derivatives, for example spirodiclofen, spiromesifen or spirotetramat;
M.24 Mitochondrial complex IV electron transport inhibitors, for example
M.24A phosphine such as aluminium phosphide, calcium phosphide, phosphine or zinc phosphide, or M.24B cyanide.
M.25 Mitochondrial complex II electron transport inhibitors, such as beta-ketonitrile derivatives, for example cyenopyrafen or cyflumetofen;
M.28 Ryanodine receptor-modulators from the class of diamides, as for example flubendiamide, chloranthraniliprole (rynaxypyr®), cyanthraniliprole (cyazypyr®), or the phthalamide compounds
M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide, or the compound
M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate;
M.UN.X insecticidal active compounds of unknown or uncertain mode of action, as for example azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, or the compound
M.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound
M.X.3: 11-(4-chloro-2,6-dimethylphenyl)-12-hydroxy-1,4-dioxa-9-azadispiro[4.2.4.2]-tetradec-11-en-10-one, or the compound
M.X.4: 3-(4′-fluoro-2,4-dimethylbiphenyl-3-yl)-4-hydroxy-8-oxa-1-azaspiro[4.5]dec-3-en-2-one, or the compound
M.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, I-1582).

The commercially available compounds of the group M listed above may be found in The Pesticide Manual, 15th Edition, C. D. S. Tomlin, British Crop Protection Council (2011) among other publications.

The phthalamides M.28.1 and M.28.2 are both known from WO 2007/101540. The anthranilamide M.28.3 has been described in WO2005/077943. The hydrazide compound M.28.4 has been described in WO 2007/043677. The quinoline derivative flometoquin is shown in WO2006/013896. The aminofuranone compounds flupyradifurone is known from WO 2007/115644. The sulfoximine compound sulfoxaflor is known from WO2007/149134. The isoxazoline compound M.X.1 has been described in WO2005/085216. The pyripyropene derivative M.X.2 has been described in WO 2006/129714. The spiroketal-substituted cyclic ketoenol derivative M.X.3 is known from WO2006/089633 and the biphenyl-substituted spirocyclic ketoenol derivative M.X.4 from WO2008/067911. Finally triazoylphenylsulfide like M.X.5 have been described in WO2006/043635 and biological control agents on basis of bacillus firmus in WO2009/124707.

The following list of active fungicidal substances, in conjunction with which the compounds according to the invention can be used, is intended to illustrate the possible combinations but does not limit them:
F.I) Respiration Inhibitors
F.I-1) Inhibitors of complex III at Qo site (e.g. strobilurins)
strobilurins: azoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, methyl (2-chloro-5[1-(3-methylbenzyloxyimino)ethyl]benzyl)carbamate and 2(2-(3-(2,6-dichlorophenyl)-1-methyl-allylideneaminooxymethyl)-phenyl)-2-methoxyimino-N methyl-acetamide;
oxazolidinediones and imidazolinones: famoxadone, fenamidone;
F.I-2) Inhibitors of complex II (e.g. carboxamides):
carboxanilides: benodanil, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′trifluorobiphenyl-2 yl)-3-difluoromethyl-1-methyl-1H-pyrazole-4 carboxamide, N-(4′-trifluoromethylthiobiphenyl-2-yl)-3 difluoromethyl-1-methyl-1H pyrazole-4-carboxamide and N-(2-(1,3,3-trimethyl-butyl)-phenyl)-1,3-dimethyl-5 fluoro-1H-pyrazole-4 carboxamide;
F.I-3) Inhibitors of complex III at Qi site: cyazofamid, amisulbrom;
F.I-4) Other respiration inhibitors (complex I, uncouplers)
diflumetorim; tecnazen; ferimzone; ametoctradin; silthiofam;
nitrophenyl derivates: binapacryl, dinobuton, dinocap, fluazinam, nitrthal-isopropyl, organometal compounds: fentin salts, such as fentin-acetate, fentin chloride or fentin hydroxide;
F.II) Sterol biosynthesis inhibitors (SBI fungicides)
F.II-1) C14 demethylase inhibitors (DMI fungicides, e.g. triazoles, imidazoles)
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole;
imidazoles: imazalil, pefurazoate, oxpoconazole, prochloraz, triflumizole;
pyrimidines, pyridines and piperazines: fenarimol, nuarimol, pyrifenox, triforine;
F.II-2) Delta14-reductase inhitors (Amines, e.g. morpholines, piperidines)
morpholines: aldimorph, dodemorph, dodemorph-acetate, fenpropimorph, tridemorph;
piperidines: fenpropidin, piperalin;
spiroketalamines: spiroxamine;
F.II-3) Inhibitors of 3-keto reductase: hydroxyanilides: fenhexamid;
F.III) Nucleic acid synthesis inhibitors
F.III-1) RNA, DNA synthesis
phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;
F.III-2) DNA topisomerase inhibitors: oxolinic acid;
F.III-3) Nucleotide metabolism (e.g. adenosin-deaminase)
hydroxy (2-amino)-pyrimidines: bupirimate;
F.IV) Inhibitors of cell division and or cytoskeleton
F.IV-1) Tubulin inhibitors: benzimidazoles and thiophanates: benomyl, carbendazim, fuberidazole, thiabendazole, thiophanate-methyl;
triazolopyrimidines: 5-chloro-7(4-methylpiperidin-1-yl)-6-(2,4,6-trifluorophenyl)-[1,2,4]triazolo[1,5 a]pyrimidine
F.IV-2) Other cell division inhibitors
benzamides and phenyl acetamides: diethofencarb, ethaboxam, pencycuron, fluopicolide, zoxamide;
F.IV-3) Actin inhibitors: benzophenones: metrafenone;
F.V) Inhibitors of amino acid and protein synthesis
F.V-1) Mmethionine synthesis inhibitors (anilino-pyrimidines)
anilino-pyrimidines: cyprodinil, mepanipyrim, nitrapyrin, pyrimethanil;
F.V-2) Protein synthesis inhibitors (anilino-pyrimidines)
antibiotics: blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors (e.g. anilino-pyrimidines)
dicarboximides: fluoroimid, iprodione, procymidone, vinclozolin;
phenylpyrroles: fenpiclonil, fludioxonil;
F.VI-2) G protein inhibitors: quinolines: quinoxyfen;
F.VII) Lipid and membrane synthesis inhibitors
F.VII-1) Phospholipid biosynthesis inhibitors
organophosphorus compounds: edifenphos, iprobenfos, pyrazophos;
dithiolanes: isoprothiolane;
F.VII-2) Lipid peroxidation
aromatic hydrocarbons: dicloran, quintozene, tecnazene, tolclofos-methyl, biphenyl, chloroneb, etridiazole;
F.VII-3) Carboxyl acid amides (CAA fungicides)
cinnamic or mandelic acid amides: dimethomorph, flumorph, mandiproamid, pyrimorph;
valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acides
carbamates: propamocarb, propamocarb-hydrochlorid
F.VIII) Inhibitors with Multi Site Action
F.VIII-1) Inorganic active substances: Bordeaux mixture, copper acetate, copper hydroxide, copper oxychloride, basic copper sulfate, sulfur;
F.VIII-2) Thio- and dithiocarbamates: ferbam, mancozeb, maneb, metam, methasulphocarb, metiram, propineb, thiram, zineb, ziram;
F.VIII-3) Organochlorine compounds (e.g. phthalimides, sulfamides, chloronitriles):
anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;
F.VIII-4) Guanidines: guanidine, dodine, dodine free base, guazatine, guazatine-acetate, iminoctadine, iminoctadine-triacetate, iminoctadine-tris(albesilate);
F.VIII-5) Ahtraquinones: dithianon;
F.IX) Cell wall synthesis inhibitors
F.IX-1) Inhibitors of glucan synthesis: validamycin, polyoxin B;
F.IX-2) Melanin synthesis inhibitors: pyroquilon, tricyclazole, carpropamide, dicyclomet, fenoxanil;
F.X) Plant defence inducers
F.X-1) Salicylic acid pathway: acibenzolar-S-methyl;
F.X-2) Others: probenazole, isotianil, tiadinil, prohexadione-calcium;
phosphonates: fosetyl, fosetyl-aluminum, phosphorous acid and its salts;
F.XI) Unknown mode of action:
bronopol, chinomethionat, cyflufenamid, cymoxanil, dazomet, debacarb, diclomezine, difenzoquat, difenzoquat-methylsulfate, diphenylamin, flumetover, flusulfamide, flutianil, methasulfocarb, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, 3-[5-(4-methyl-phenyl)-2,3-dimethyl-isoxazolidin-3-yl]-pyridine, 5-amino-2-isopropyl-3-oxo-4-ortho-tolyl-2,3-dihydro-pyrazole-1 carbothioic acid S-allyl ester, N-(6-methoxy-pyridin-3-yl)cyclopropane-carboxylic acid amide, 5-chloro-1(4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;
F.XI) Growth regulators:
abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid , trinexapac-ethyl and uniconazole;
F.XII) Biological control agents
antifungal biocontrol agents: Bacillus substilis strain with NRRL No. B-21661 (e.g. RHAPSO-DY®, SERENADE® MAX and SERENADE® ASO from AgraQuest, Inc., USA.), Bacillus pumilus strain with NRRL No. B-30087 (e.g. SONATA® and BALLAD® Plus from AgraQuest, Inc., USA), Ulocladium oudemansii (e.g. the product BOTRY-ZEN from BotriZen Ltd., New Zealand), Chitosan (e.g. ARMOUR-ZEN from BotriZen Ltd., New Zealand).

### Applications

In the following sections the term “compound of formula (I)” includes compounds of formula (I), salts and N-oxides thereof.

The invertebrate pest, i.e. the insects, arachnids and nematodes, the plant, soil or water in which the plant is growing can be contacted with the compounds of formula (I) or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula (I) or the pesticidal compositions comprising them may be used to protect growing plants and crops from attack or infestation by invertebrate pests, especially insects, acaridae or arachnids by contacting the plant/crop with a pesticidally effective amount of compounds of formula (I). The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with a insecticidally effective amount of the active compounds. The application can be carried out both before and after the infection of the plants, plant propagation materials, such as seeds, soil, surfaces, materials or rooms by the insects.

The present invention also includes a method of combating animal pests which comprises contacting the animal pests, their habit, breeding ground, food supply, cultivated plants, seed, soil, area, material or environment in which the animal pests are growing or may grow, or the materials, plants, seeds, soils, surfaces or spaces to be protected from animal attack or infestation with a pesticidally effective amount of a mixture of at least one active compound (I).

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula (I) may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8., Protein Eng Des Sel. 2004 January; 17(1):57-66, Nat Protoc. 2007; 2(5):1225-35., Curr Opin Chem Biol. 2006 October; 10(5):487-91. Epub 2006 Aug. 28., Biomaterials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxyphenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as ä-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are disclosed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for example oil crops that produce health-promoting long-chain omega-3 fatty acids or unsaturated omega-9 fatty acids (e.g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e.g. Amflora® potato).

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of formula I are effective through both contact (via soil, glass, wall, bed net, carpet, plant parts or animal parts), and ingestion (bait, or plant part).

The compounds of the invention may also be applied against non-crop insect pests, such as ants, termites, wasps, flies, mosquitos, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxy-methylcyclohexyl) acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

### Seed Treatment

The compounds of formula (I) are also suitable for the treatment of seeds in order to protect the seed from insect pest, in particular from soil-living insect pests and the resulting plant's roots and shoots against soil pests and foliar insects.

The compounds of formula (I) are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The invention therefore provides a method for the protection of seeds from insects, in particular from soil insects and of the seedling's roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting. The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:
A Soluble concentrates (SL, LS)
D Emulsions (EW, EO, ES)
E Suspensions (SC, OD, FS)
F Water-dispersible granules and water-soluble granules (WG, SG)
G Water-dispersible powders and water-soluble powders (WP, SP, WS)
H Gel-Formulations (GF)
I Dustable powders (DP, DS)
   Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typcially, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

An example of a gelling agent is carrageen (Satiagel®).

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### Animal Health

The compounds of formula (I) or veterinarily acceptable salts thereof are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula I or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

Compounds of formula (I) or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula (I) or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula (I) or veterinarily acceptable salts thereof and compositions comprising them are suitable for systemic and/or non-systemic control of ecto- and/or endoparasites. They are active against all or some stages of development.

The compounds of formula (I) are especially useful for combating ectoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliompa hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucllia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor vanabilis, Amblyomma americanum, Ambryomma maculatum, Ornithodorus hermsi, Ornithodorus turicata and parasitic mites (Mesostigmata), e.g. Ornithonyssus Bacoti and Dermanyssus gallinae,

Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatonia spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicphalobus spp,

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinana spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascardia galli, Parascaris equorum, Enterobius vermiculans (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wucherera spp., Brugia spp., Onchocerca spp., Dirofilari spp.a, Dipetalonema spp., Setaria spp., Elaeophora spp., Spirocerca lupi, and Habronema spp.,

Thorny headed worms (Acanthocephala), e.g. Acanthocepha/us spp., Macracanthorhynchus hirudinaceus and Oncicola spp,

Planarians (Plathelminthes):

Flukes (Trematoda), e.g. Faciola spp., Fascioloides magna, Paragonimus spp., Dicrocoelium spp., Fasciolopsis busk, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I) and compositions containing them are particularly useful for the control of pests from the orders Diptera, Siphonaptera and Ixodida.

Moreover, the use of the compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, compounds of formula (I) may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the compound of formula I may be transdermally administered to animals. For parenteral administration, the dosage form chosen should provide the animal with 0.01 mg/kg to 100 mg/kg of animal body weight per day of the compound of formula (I).

The compounds of formula (I) may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are, e.g.:

Solutions such as oral solutions, concentrates for oral administration after dilution, solutions for use on the skin or in body cavities, pouring-on formulations, gels;

Emulsions and suspensions for oral or dermal administration; semi-solid preparations;

Formulations in which the active compound is processed in an ointment base or in an oil-in-water or water-in-oil emulsion base;

Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 per cent by weight, preferably from 0.1 to 65 per cent by weight, more preferably from 1 to 50 per cent by weight, most preferably from 5 to 40 per cent by weight.

Preparations which are diluted before use contain the compounds acting against ectoparasites in concentrations of 0.5 to 90 per cent by weight, preferably of 1 to 50 per cent by weight.

Furthermore, the preparations comprise the compounds of formula I against endoparasites in concentrations of 10 ppm to 2 per cent by weight, preferably of 0.05 to 0.9 per cent by weight, very particularly preferably of 0.005 to 0.25 per cent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

### EXAMPLES

The invention is now illustrated in further details by the following examples, without imposing any limitation thereto.

### C. Compound Examples

Compounds can be characterized e.g. by coupled High Performance Liquid Chromatography/mass spectrometry (HPLC/MS), by ¹H-NMR and/or by their melting points.

Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

¹H-NMR, respectively ¹³C-NMR: The signals are characterized by chemical shift (ppm) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, q=quartett, t=triplett, d=doublet and s=singulett.

### C.1 Compound Examples of Table B

The compound examples of Table B, 1-1 and 1-2, correspond to formula (I):

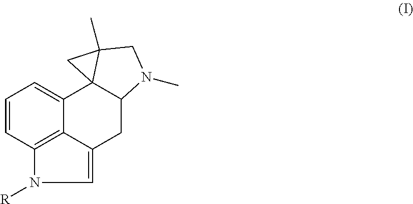

wherein R of each compound example is defined below.

TABLE B

| | | |
|---|---|---|
| Compound | | HPLC-MS: |
| Ex. | R | Rₜ (min) and [M + H] |

| | | | |
|---|---|---|---|
| 1-1 | H | 1.977 | 238.90 |
| 1-2 | C(═O)—OCH₃ | 2.325 | 296.90 |

### S. Synthesis Examples

### S.1 Synthesis of N-methylcarbamoyl-(1aR,3aR,9bR)-1a,2,3,3a,4,6-hexahydro-1a,3-dimethyl-1H-cycloprop[c]indolo[4,3-ef]indole (cycloclavin-N-methylcarbamat, compound 1-2 of table B)

A solution of cycloclavin (21 mg), 1,4-diazabicyclo[2,2,2]octane (1 mg, 10 mol %) and dimethyl carbonate (0.82 mL) in DMF (0.4 mL) was kept at 95° C. for 1.5 h. Further dimethyl carbonate (0.6 mL) was added and heated to 95° C. for another 14 h. After cooling, water was added and the mixture was extracted with ethyl acetate. The aqueous layer was extracted with ethyl acetate twice and combined organic layers were washed with water twice. The organic layer was dried (Na₂SO₄) and evaporated in vacuum to yield the title compound (10 mg, 45%).

Characterization by HPLC-MS: 2.325 min, m/z=296.90

Characterization by ¹H-NMR (500 MHz, CDCl₃):

δ[delta]=0.42 (d, 1H), 1.63 (d, 1H), 1.67 (s, 3H), 2.40 (s, 3H), 2.42 (m, 1H), 2.51 (m, 1H), 2.70 (m, 1H), 3.06 (dd, 1H) 3.15 (d, 1H), 4.05 (s, 3H), 6.97 (d, 1H), 7.21 (m, 1H), 7.30 (m, 1H), 7.79 (br. s, 1H) ppm.

### B. Biological Examples

The activity of compounds of formula (I) could be demonstrated and evaluated in biological tests described in the following.

If not otherwise specified the test solutions are prepared as follow:

The active compound is dissolved at the desired concentration in a mixture of 1:1 (vol:vol) distilled water:acteon. The test solution is prepared at the day of use and in general at concentrations of ppm (wt/vol).

B.1 Cotton Aphid (Aphis gossypi)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone: 50% water (v/v). A nonionic surfactant (Kinetic®) was included in the solution at a volume of 0.01% (v/v).

Cotton plants at the cotyledon stage were infested with aphids prior to treatment by placing a heavily infested leaf from the main aphid colony on top of each cotyledon. Aphids were allowed to transfer overnight to accomplish an infestation of 80-100 aphids per plant and the host leaf was removed. The infested plants were then sprayed by an automated electrostatic plant sprayer equipped with an atomizing spray nozzle. The plants were dried in the sprayer fume hood, removed from the sprayer, and then maintained in a growth room under fluorescent lighting in a 24-hr photoperiod at 25° C. and 20-40% relative humidity. Aphid mortality on the treated plants, relative to mortality on untreated control plants, was determined after 5 days.

In this test, the compounds 1-1 and 1-2, respectively, at 300 ppm showed a mortality of at least 75% in comparison with untreated controls.

B.2 Cowpea Aphid (aphis craccivora)

Potted cowpea plants colonized with approximately 100-150 aphids of various stages were sprayed after the pest population has been recorded. Population reduction was assessed after 24, 72, and 120 hours.

In this test, the compounds 1-1 and 1-2 respectively, at 300 ppm showed a mortality of at least 75% in comparison with untreated controls.

B.3 Diamond Back Moth (plutella xylostella)

Leaves of Chinese cabbage were dipped in test solution and air-dried. Treated leaves were placed in petri dished lined with moist filter paper. Mortality was recorded 24, 72, and 120 hours after treatment.

In this test, the compound 1-2, at 300 ppm showed a mortality of at least 75% in comparison with untreated controls.

B.4 Orchid Thrips (dichromothrips corbetti)

Dichromothrips corbetti adults used for bioassay are obtained from a colony maintained continuously under laboratory conditions. For testing purposes, the test compound is diluted to a concentration of 300 ppm (wt compound: vol diluent) in a 1:1 mixture of acetone:water (vol:vol), plus 0.01% vol/vol Kinetic® surfactant.

Thrips potency of each compound is evaluated by using a floral-immersion technique. Plastic petri dishes are used as test arenas. All petals of individual, intact orchid flowers are dipped into treatment solution and allowed to dry. Treated flowers are placed into individual petri dishes along with 10-15 adult thrips. The petri dishes are then covered with lids. All test arenas are held under continuous light and a temperature of about 28° C. for duration of the assay. After 4 days, the numbers of live thrips are counted on each flower, and along inner walls of each petri dish. The level of thrips mortality is extrapolated from pre-treatment thrips numbers.

In this test, the compound 1-2, at 300 ppm showed a mortality of at least 75% in comparison with untreated controls.

B.5 Silverleaf Whitefly (Bemisia argentifolii)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic( ) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compound 1-1, at 300 ppm showed a mortality of at least 75% in comparison with untreated controls.

B.6 Red spider Mite (Tetranychus kanzawai)

The active compound was dissolved at the desired concentration in a mixture of 1:1 (v/v) distilled water:acetone. A surfactant (Alkamuls® EL 620) was added at the rate of 0.1% (v/v).

Potted cowpea beans of 7-10 days of age were cleaned with tap water and sprayed with 5 ml of the test solution using air driven hand atomizer. The treated plants were allowed to air dry and afterwards inculated with 20 or more mites by clipping a cassava leaf section with known mite population. Treated plants were placed inside a holding room at about 25-27° C. and about 50-60% relatice humidity.

ortality by counting the live mites 72 HAT. Percent mortality was assessed after 72 h.

In this test, the compound 1-2, at 300 ppm showed a mortality of at least 75% in comparison with untreated controls.

B.7 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, the compound 1-1, at 2500 ppm showed a mortality of at least 75% in comparison with untreated controls.

B.8 Green Peach Aphid (Myzus persicae)

The active compounds are formulated in cyclohexanone as a 10,000 ppm solution supplied in tubes. The tubes are inserted into an automated electrostatic sprayer equipped with an atomizing nozzle and they served as stock solutions for which lower dilutions are made in 50% acetone: 50% water (v/v). A nonionic surfactant (Kinetic®) is included in the solution at a volume of 0.01% (v/v).

Bell pepper plants at the first true-leaf stage are infested prior to treatment by placing heavily infested leaves from the main colony on top of the treatment plants. Aphids are allowed to transfer overnight to accomplish an infestation of 30-50 aphids per plant and the host leaves are removed. The infested plants are then sprayed by an automated electrostatic plant sprayer equipped with an atomizing spray nozzle. The plants are dried in the sprayer fume hood, removed, and then maintained in a growth room under fluorescent lighting in a 24-hr photoperiod at about 25° C. and about 20-40% relative humidity. Aphid mortality on the treated plants, relative to mortality on untreated control plants, is determined after 5 days.

In this test, the compounds 1-1 and 1-2, respectively, at 300 ppm showed a mortality of at least 75% in comparison with untreated controls.

B.9 Rice Green Leafhopper (Nephotettix virescens)

Rice seedlings are cleaned and washed 24 hours before spraying. The active compounds are formulated in 50:50 acetone:water (vol:vol), and 0.1% vol/vol surfactant (EL 620) is added. Potted rice seedlings are sprayed with 5 ml test solution, air dried, placed in cages and inoculated with 10 adults. Treated rice plants are kept at about 28-29° C. and relative humidity of about 50-60%. Percent mortality is recorded after 72 hours.

In this test, the compound 1-1, at 300 ppm showed a mortality of at least 75% in comparison with untreated controls.

B.10 Striped Flea Beetle (Phylotretta striolata)

The active compound is dissolved at the desired concentration in a mixture of 1:1 (vol:vol) distilled water:acetone. Add surfactant (Alkamuls® EL620) at the rate of 0.1% (vol/vol). The test solution is prepared at the day of use.

Leaves of Chinese cabbage were dipped in test solution and air-dried. Treated leaves were placed in pertri dishes lined with moist filter paper and inoculated with 20 adults. Mortality was recorded 72 hours after treatment. Feeding damage were also recorded using scale of 0-100%.

In this test, the compound 1-2, at 300 ppm showed a mortality of at least 75% in comparison with untreated controls.

### BA. Animal Health

### General Test Conditions of Animal Health Glass Vial Contact Assays

If not otherwise specified, the tests were conducted as glass vial contact assays. Glass vials (20 ml scintillation vials) were used. Treatment solutions were mixed with technical grade chemicals diluted in acetone. Treatment solutions needed for the assays included generally 1 and 10 ppm (0.01 and 0.1 μg/cm², respectively), but optionally also 100 and/or 1000 ppm for first tier vials. As commercial standard, alphacypermethrin, was run at 1 ppm. As solvent control, acetone was used for the assay. Treatment solution was pipetted into the bottom of each vial. Each vial was turned on its side and placed onto a commercial grade hot dog roller without applying heat. The uncapped vials were allowed to roll to allow for the acetone treatment to vent off. After drying, the vials were placed into the compartmented vial shipping boxes. The workstation was prepared by chilling the table and plastic Petri dishes with the inside wall coated with Fluon. A weigh boat of 10% sugar water saturated cotton dental pellets was also prepared. The animal pests were collected into a tube with a rechargeable insect vacuum. The tube of animal pests was placed in a laboratory refrigerator until the animal pests were incapacitated. The animal pests were emptied into chilled Petri dish. A small cotton dental pellet was soaked in water or in 10 wt % sugar water, whereas the excess solution was gently squeezed out. The cotton dental pellet was placed into the bottom of each vial. For the test, the animal pests were added to each vial and then the cap was loosely put on the vial to allow for ventilation. The test vials were hold at ambient room temperature in compartmented boxes. In general, the animal pests were observed for incapacitation at least at 4, 24, and 48 hours after infestation, or for a longer period if required. Mortality was defined as an insect incapable of coordinated movement when agitated.

### BA.2.a Larval Mosquito Water Treatment Assay

The assay was conducted in 6-well polystyrene plates using one plate per treatment rate. Stock solutions were prepared at 100 and 1000 ppm. Screen rates were at 1 and 10 ppm. Distilled water was added to each well, control wells were treated with acetone. Temephos (Abate technical) was used as the standard at 0.1 ppm. Ten late third-instar yellowfever mosquito larvae (Aedes aegypti) in water were added to each well. One drop of liver powder solution (6 g in 100 ml distilled water) was added to each well as a food source daily. Plates were maintained at 22-25° C. and 25-50% RH (relative humidity) and observed daily for dead larvae and pupae at 1, 2, 3, and 5 days after treatment. Dead larvae and all pupae were removed daily. Mortality was defined as an insect incapable of coordinated movement when agitated.

In this first tier test, the compound 1-1, at 10 ppm showed after 5 DAT (days after treament) a mortality of at least 60% in comparison with untreated controls.

1-17. (canceled)

18. A method for controlling invertebrate pests comprising contacting the invertebrate pests, their habitat, breeding ground, food supply, plant, seed, soil, area, material or environment in which the invertebrate pests are growing or may grow, or the materials, plants, seed, soils, surfaces or spaces to be protected from attack or infestation with a pesticidally effective amount of at least one indole alkaloid compound of the general formula (I), or a salt or an N-oxide thereof 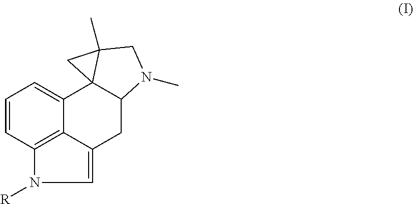
wherein the symbol in formula (I) has the following meanings:
R is R′, C(═O)R′, C(═O)OR′, C(═O)NR′₂, C(═S)R′, C(═S)OR′, C(═S)NR′₂, C(═NR″)R′, C(═NR″)NR′₂, S(O)R′, S(O)ₙNR′₂, Si(R′″)₃ or NR′₂;
each R′ is selected from the group consisting of hydrogen, C₁-C₆-alkyl, C₂-C₆-alkenyl, C₂-C₆-alkynyl, wherein the carbon atoms of the aforementioned aliphatic radicals are unsubstituted or substituted with one or more cyano, nitro, nitroso,
C₃-C₈-cycloalkyl, C₃-C₈-cycloalkenyl, each unsubstituted or substituted with one or more R²,
phenyl, unsubstituted or substituted with up to five R²,
and a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R″ is independently selected from the group consisting of hydrogen, C₁-C₆ alkyl, C₂-C₆ alkenyl, C₂-C₆ alkynyl, each unsubstituted or substituted with one or more R¹,
C₃-C₈ cycloalkyl, C₃-C₈ cycloalkenyl, each unsubstituted or substituted with one or more R²,
phenyl, unsubstituted or substituted with up to five R²,
and a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R′″ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, haloalkoxyalkyl,
phenyl, and a 3-, 4-, 5-, -6 or 7-membered saturated, partially unsaturated or aromatic heterocyclic ring containing 1, 2 or 3 heteroatoms or heteroatom groups selected from N, O, S, NO, SO, SO₂;
each R¹ is independently selected from the group consisting of halogen, cyano, azido, nitro, SCN, SF₅
phenyl, unsubstituted or substituted with up to five R²,
a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
C═(O)R³, C(═O)OR⁴, C(═O)NR⁵₂, C(═S)R³, C(═S)OR⁴, C(═S)NR⁵₂, C(═NR⁶)R³, C(═NR⁶)NR⁵₂, S(═O)ₙR⁴, S(═O)ₙNR⁵₂, Si(R⁷)₃, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂;
each R² is independently selected from the group consisting of halogen, cyano, azido, nitro, SCN, SF₅,
C₁-C₆-alkyl, C₂-C₆-alkenyl, C₂-C₆-alkynyl, wherein the carbon atoms of the aforementioned aliphatic radicals are unsubstituted or substituted with one or more R^{b},
C₃-C₈-cycloalkyl, C₃-C₈-cycloalkenyl, each unsubstituted or substituted with one or more R^{e},
phenyl, unsubstituted or substituted with up to five R^{a},
a 3-, 4-, 5-, 6- or 7-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
C═(O)R³, C(═O)OR⁴, C(═O)NR⁵₂, C(═S)R³, C(═S)OR⁴, C(═S)NR⁵₂, C(═NR⁶)R³, C(═NR⁶)NR⁵₂, S(═O)ₙR⁴, S(═O)ₙNR⁵₂, Si(R⁷)₃, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, and NR⁵—CO—OR⁴;
each R³, is independently selected from the group consisting of hydrogen, cyano, C₁-C₆ alkyl, C₂-C₆ alkenyl, C₂-C₆ alkynyl, wherein the carbon atoms of the aforementioned aliphatic radicals are unsubstituted or substituted with one or more R^{b},
C₃-C₈ cycloalkyl or C₃-C₈ cycloalkenyl, wherein the carbon atoms of the aforementioned cycloaliphatic radicals are unsubstituted or substituted with one or more R^{a},
phenyl unsubstituted or substituted with up to 5 R^{a},
and a 3-, 4-, 5-, 6- or 7-membered saturated, partially unsaturated or aromatic heterocyclic ring containing 1, 2 or 3 heteroatoms or heteroatom groups selected from N, O, S, NO, SO, SO₂, wherein the aforementioned ring is unsubstituted or substituted with one or more R^{a};
each R⁴, R⁵ is independently selected from the group consisting of hydrogen, C₁-C₆ alkyl, C₂-C₆ alkenyl, C₂-C₆ alkynyl, wherein the carbon atoms of the aforementioned aliphatic radicals are unsubstituted or substituted with one or more R^{b},
C₃-C₈ cycloalkyl or C₃-C₈ cycloalkenyl, wherein the carbon atoms of the aforementioned cycloaliphatic radicals are unsubstituted or substituted with one or more R^{a},
phenyl unsubstituted or substituted with up to 5 R^{a},
and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO, and SO₂, wherein the aforementioned ring is unsubstituted or substituted with one or more R^{a};
each R⁶ is independently selected from the group consisting of hydrogen, C₁-C₆ alkyl, C₂-C₆ alkenyl, C₂-C₆ alkynyl, wherein the carbon atoms of the aforementioned aliphatic radicals are unsubstituted or substituted with one or more R^{b},
C₃-C₈ cycloalkyl or C₃-C₈ cycloalkenyl, wherein the carbon atoms of the aforementioned cycloaliphatic radicals are unsubstituted or substituted with one or more R^{a},
phenyl unsubstituted or substituted with up to 5 R^{a},
and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO, and SO₂, wherein the aforementioned ring is unsubstituted or substituted with one or more R^{a} or OR⁴;
each R⁷ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, C₁-C₆ haloalkoxyalkyl,
phenyl, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO, and SO₂;
each R^{a} is independently selected from the group consisting of halogen, cyano, azido, nitro, OH, SH, —SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tert-butyldimethylsilyl, C₁-C₆ alkyl, C₁-C₆ haloalkyl, C₂-C₆ halkenyl, C₂-C₆ haloalkenyl, C₂-C₆ alkynyl, C₂-C₆ haloalkynyl, each unsubstituted or substituted with one or two radicals selected from C₁-C₄ alkoxy and C₁-C₄ haloalkoxy,
C₃-C₈ cycloalkyl, C₃-C₈ cycloalkenyl, C₃-C₈ halocycloalkyl, C₃-C₈ halocycloalkenyl, each unsubstituted or substituted with one or two radicals selected from the group consisting of C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
each R^{b} is independently selected from the group consisting of halogen, cyano, azido, nitro, OH, SH, —SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tert-butyldimethylsilyl,
C₃-C₈ cycloalkyl, C₃-C₈ cycloalkenyl, C₃-C₈ halocycloalkenyl, C₃-C₈ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl; and
each n is independently 1 or 2.

19. The method of claim 18, wherein
R is R′, C(═O)R′, C(═O)OR′, C(═O)NR′₂, C(═S)R′, C(═S)OR′, C(═S)NR′₂, C(═NR″)R′, C(═NR″)NR′₂, S(O)ₙR′, S(O)ₙNR′₂ or NR′₂;
each R′ is independently selected from the group consisting of hydrogen, C₁-C₆-alkyl, C₂-C₆-alkenyl, wherein the carbon atoms of the aforementioned aliphatic radicals are unsubstituted or substituted with one or more R¹, cyano,
C₃-C₈-cycloalkyl, C₅-C₆-cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R″ is independently selected from the group consisting of hydrogen,
C₁-C₆ alkyl, C₂-C₆ alkenyl, each unsubstituted or substituted with one or more R¹,
C₃-C₈ cycloalkyl, C₅-C₆ cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R′″ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl and phenyl;
each R¹ is independently halogen, cyano,
phenyl, unsubstituted or substituted with up to five R²,
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, or NR⁵—CO—NR⁵₂;
each R² is independently halogen, cyano,
C₁-C₆-alkyl, C₂-C₆-alkenyl, wherein the carbon atoms of the aforementioned aliphatic radicals are unsubstituted or substituted with one or more R^{b},
C₃-C₈-cycloalkyl, C₅-C₆-cycloalkenyl, each unsubstituted or substituted with one or more R^{a},
phenyl, unsubstituted or substituted with up to five R^{a},
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, or NR⁵—CO—OR⁴;
each R³, R⁴, R⁵ is independently hydrogen, C₁-C₆ alkyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from N, O, S, NO, SO, and SO₂, wherein the aforementioned ring is unsubstituted or substituted with one or more R^{a};
each R⁶ is independently hydrogen, C₁-C₆ alkyl, C₂-C₆ alkenyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from the group consisting of N, O, S, NO, SO, SO₂, wherein the aforementioned ring is unsubstituted or substituted with one or more R^{a} or OR⁴;
each R⁷ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl,
phenyl, and a 3-, 4-, 5- or 6-membered saturated, partially unsaturated or aromatic heterocyclic ring containing 1, 2 or 3 heteroatoms or heteroatom groups selected from N, O, S, NO, SO, SO₂;
each R^{a} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆ alkyl, C₁-C₆ haloalkyl, C₂-C₆ alkenyl, C₂-C₆ haloalkenyl, each unsubstituted or substituted with one or two radicals selected from C₁-C₄ alkoxy and C₁-C₄ haloalkoxy,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
each R^{b} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl;
each n is independently 1 or 2.

20. The method of claim 18, wherein the symbols in formula (I) have the following meanings:
R is R′, NR′₂, C(═O)R′, C(═O)OR′, or C(═O)NR′₂;
each R′ is independently hydrogen;
C₁-C₆-alkyl, C₂-C₆-alkenyl, each substituted with one or more R¹,
phenyl, substituted with one or two R²,
or is selected from the group consisting of: 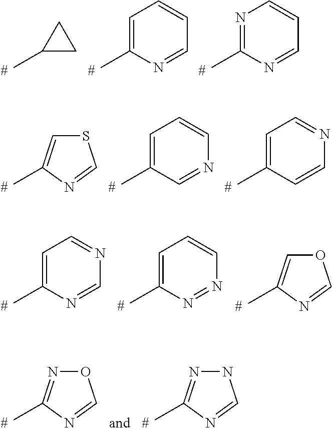
wherein each of the above ring systems is unsubstituted or substituted with one or more R²;
each R¹ is independently halogen;
each R² is independently halogen, C₁-C₆ alkyl, C₁-C₆ haloalkyl or OR⁴; and
each R⁴ is independently C₁-C₆ alkyl or C₁-C₆ haloalkyl.

21. The method of claim 18, wherein the symbols in formula (I) have the following meanings:
R is R′, NR′₂, C(═O)R′, C(═O)OR′ or C(═O)NR′₂;
each R′ is hydrogen,
C₁-C₆-alkyl, C₂-C₆-alkenyl, each substituted with one or more R¹,
phenyl, substituted with one or two R²,
or is selected from A1 to A28: 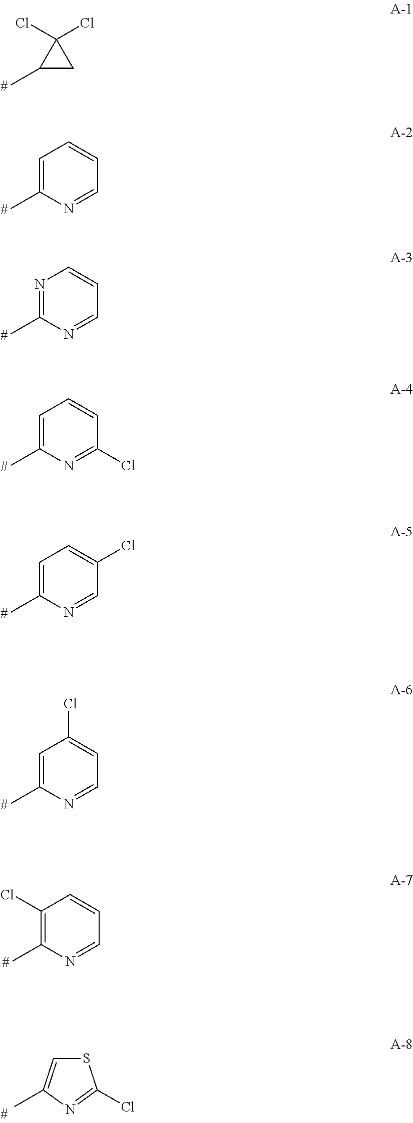 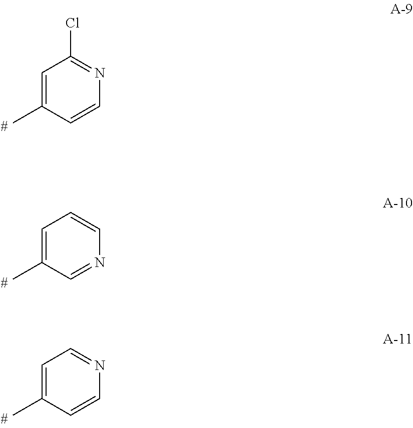 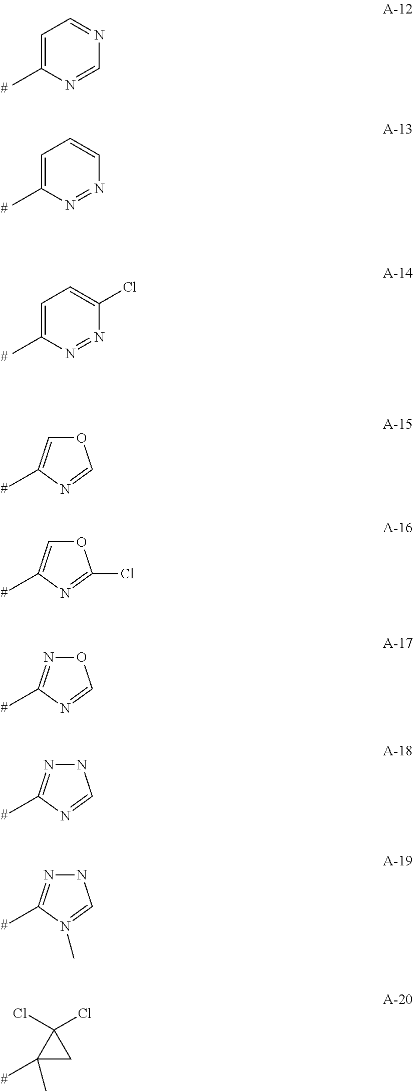 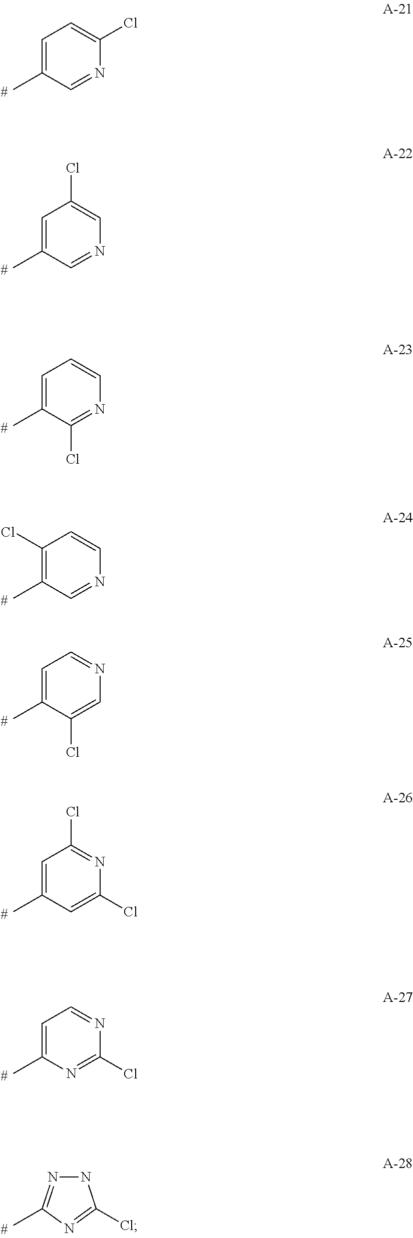
each R¹ is independently halogen;
each R² is independently halogen, C₁-C₆ alkyl, C₁-C₆ haloalkyl or OR⁴;
each R⁴ is independently C₁-C₆ alkyl or C₁-C₆ haloalkyl.

22. A compound of formula (I) or a salt or N-oxide thereof of claim 18 with the proviso that R is not H.

23. A method for preparing the compound of formula (I) of claim 22, comprising the step of reacting the compound of formula (Ia) 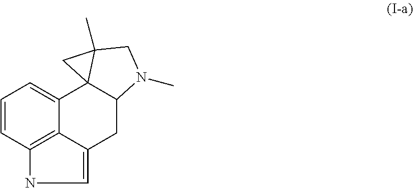 [SMILES: CN1CC2(C)CC23c2cccc4[nH]cc(c24)CC13]
with a compound of formula (II),
R-L (II)
wherein
R is defined as in formula (I) and is ≠H and
L is a leaving group,
optionally in the presence of a base.

24. An agricultural and/or veterinary composition comprising at least one compound of formula (I) of claim 22 or a salt or N-oxide thereof.

25. The composition of claim 24, further comprising at least one inert liquid and/or at least one solid carrier.

26. A method for protecting crops from attack or infestation by invertebrate pests comprising contacting the crop with a pesticidally effective amount of at least one compound of formula (I) of 18 or a salt or an N-oxide thereof.

27. A method for protecting seeds from soil insects and the seedlings' roots and shoots from soil and foliar insects comprisingcontacting the seeds before sowing and/or after pregermination with at least one compound of formula (I) of claim 18 or a salt or an N-oxide thereof.

28. A seed treated with at least one compound of formula (I) of claim 22 or a salt or an N-oxide thereof.

29. A method for treating or protecting animals against infestation or infection by parasites comprising orally, topically or parenterally administering or applying to the animals a parasiticidally effective amount of at least one compound of formula (I) of claim 18 or a salt or an N-oxide thereof.

30. A method for the preparation of a composition for treating or protecting animals against infestation or infection by parasites comprising mixing a parasiticidally effective amount of at least one compound of formula (I) of claim 22 or a salt or an N-oxide thereof and at least one solid carrier.

31. The method of claim 26, wherein
R is R′, C(═O)R′, C(═O)OR′, C(═O)NR′₂, C(═S)R′, C(═S)OR′, C(═S)NR′₂, C(═NR″)R′, C(═NR″)NR′₂, S(O)ₙR′, S(O)ₙNR′₂ or NR′₂;
each R′ is independently selected from the group consisting of hydrogen, C₁-C₆-alkyl, C₂-C₆-alkenyl, wherein the carbon atoms of the aforementioned aliphatic radicals are unsubstituted or substituted with one or more R¹, cyano,
C₃-C₈-cycloalkyl, C₅-C₆-cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R″ is independently selected from the group consisting of hydrogen,
C₁-C₆ alkyl, C₂-C₆ alkenyl, each unsubstituted or substituted with one or more R¹,
C₃-C₈ cycloalkyl, C₅-C₆ cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R′″ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl and phenyl;
each R¹ is independently halogen, cyano,
phenyl, unsubstituted or substituted with up to five R²,
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, or NR⁵—CO—NR⁵₂;
each R² is independently halogen, cyano,
C₁-C₆-alkyl, C₂-C₆-alkenyl, wherein the carbon atoms of the aforementioned aliphatic radicals are unsubstituted or substituted with one or more R^{b},
C₃-C₈-cycloalkyl, C₅-C₆-cycloalkenyl, each unsubstituted or substituted with one or more R^{a},
phenyl, unsubstituted or substituted with up to five R^{a},
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, or NR⁵—CO—OR⁴;
each R³, R⁴, R⁵ is independently hydrogen, C₁-C₆ alkyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from N, O, S, NO, SO, and SO₂, wherein the aforementioned ring is unsubstituted or substituted with one or more R^{a};
each R⁶ is independently hydrogen, C₁-C₆ alkyl, C₂-C₆ alkenyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from the group consisting of N, O, S, NO, SO, SO₂, wherein the aforementioned ring is unsubstituted or substituted with one or more R^{a} or OR⁴;
each R⁷ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl,
phenyl, and a 3-, 4-, 5- or 6-membered saturated, partially unsaturated or aromatic heterocyclic ring containing 1, 2 or 3 heteroatoms or heteroatom groups selected from N, O, S, NO, SO, SO₂;
each R^{a} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆ alkyl, C₁-C₆ haloalkyl, C₂-C₆ alkenyl, C₂-C₆ haloalkenyl, each unsubstituted or substituted with one or two radicals selected from C₁-C₄ alkoxy and C₁-C₄ haloalkoxy,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
each R^{b} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl;
each n is independently 1 or 2.

32. The method of claim 26, wherein the symbols in formula (I) have the following meanings:
R is R′, NR′₂, C(═O)R′, C(═O)OR′, or C(═O)NR′₂;
each R′ is independently hydrogen;
C₁-C₆-alkyl, C₂-C₆-alkenyl, each substituted with one or more R¹,
phenyl, substituted with one or two R²,
or is selected from the group consisting of: 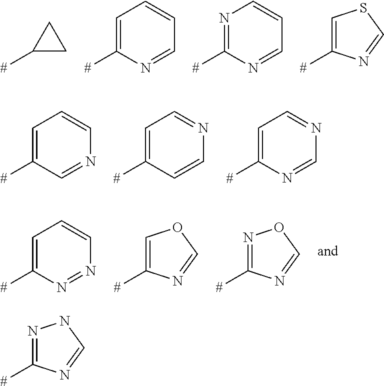
wherein each of the above ring systems is unsubstituted or substituted with one or more R²;
each R¹ is independently halogen;
each R² is independently halogen, C₁-C₆ alkyl, C₁-C₆ haloalkyl or OR⁴; and
each R⁴ is independently C₁-C₆ alkyl or C₁-C₆ haloalkyl.

33. The method of claim 26, wherein the symbols in formula (I) have the following meanings:
R is R′, NR′₂, C(═O)R′, C(═O)OR′ or C(═O)NR′₂;
each R′ is hydrogen,
C₁-C₆-alkyl, C₂-C₆-alkenyl, each substituted with one or more R¹,
phenyl, substituted with one or two R²,
or is selected from A1 to A28: 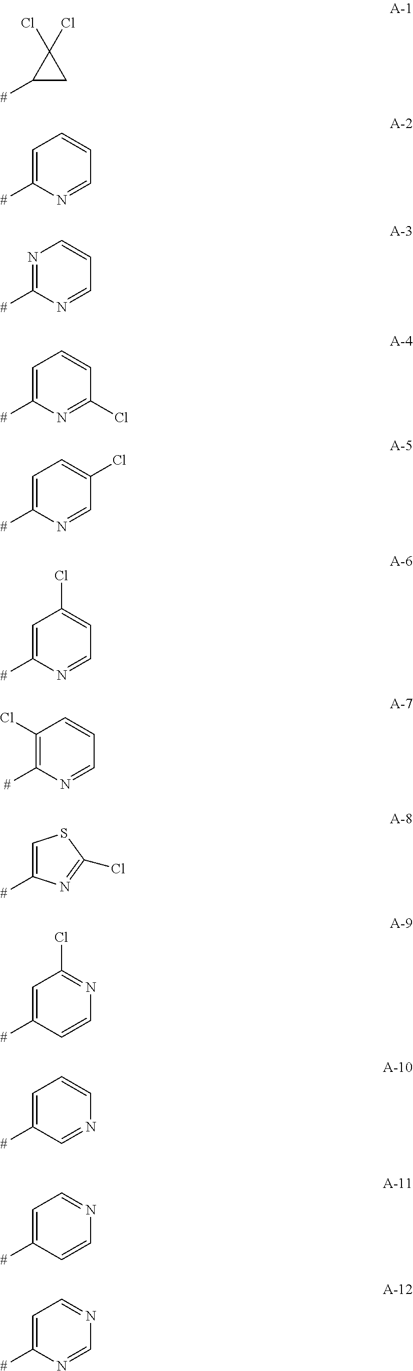 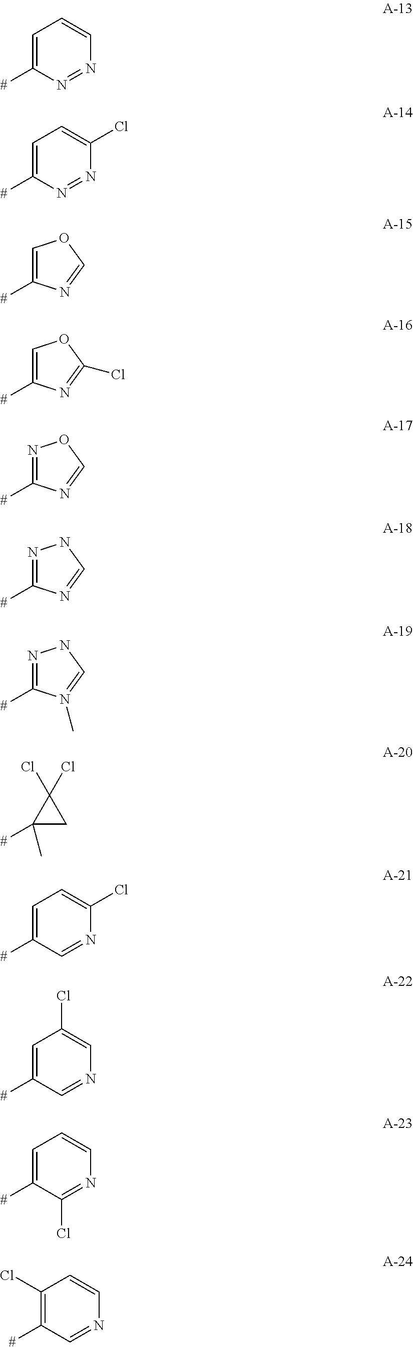 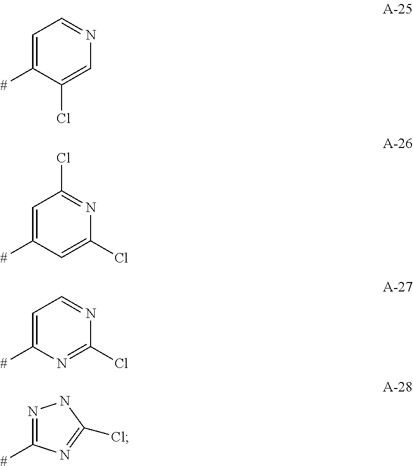
each R¹ is independently halogen;
each R² is independently halogen, C₁-C₆ alkyl, C₁-C₆ haloalkyl or OR⁴;
each R⁴ is independently C₁-C₆ alkyl or C₁-C₆ haloalkyl.

34. The method of claim 27, wherein
R is R′, C(═O)R′, C(═O)OR′, C(═O)NR′₂, C(═S)R′, C(═S)OR′, C(═S)NR′₂, C(═NR″)R′, C(═NR″)NR′₂, S(O)ₙNR′₂ or NR′₂;
each R′ is independently selected from the group consisting of hydrogen, C₁-C₆-alkyl, C₂-C₆-alkenyl, wherein the carbon atoms of the aforementioned aliphatic radicals are unsubstituted or substituted with one or more R¹, cyano,
C₃-C₈-cycloalkyl, C₅-C₆-cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R″ is independently selected from the group consisting of hydrogen,
C₁-C₆ alkyl, C₂-C₆ alkenyl, each unsubstituted or substituted with one or more R¹,
C₃-C₈ cycloalkyl, C₅-C₆ cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R′″ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl and phenyl;
each R¹ is independently halogen, cyano,
phenyl, unsubstituted or substituted with up to five R²,
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
C═(O)R³, C(═O)OR⁴, C(═O)NR⁵₂, C(═S)R³, C(═S)OR⁴, C(═S)NR⁵₂, S(═O)R⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, or NR⁵—CO—NR⁵₂;
each R² is independently halogen, cyano,
C₁-C₆-alkyl, C₂-C₆-alkenyl, wherein the carbon atoms of the aforementioned aliphatic radicals are unsubstituted or substituted with one or more R^{b},
C₃-C₈-cycloalkyl, C₅-C₆-cycloalkenyl, each unsubstituted or substituted with one or more R^{a},
phenyl, unsubstituted or substituted with up to five R^{a},
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, or NR⁵—CO—OR⁴;
each R³, R⁴, R⁵ is independently hydrogen, C₁-C₆ alkyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from N, O, S, NO, SO, and SO₂, wherein the aforementioned ring is unsubstituted or substituted with one or more R^{a};
each R⁶ is independently hydrogen, C₁-C₆ alkyl, C₂-C₆ alkenyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from the group consisting of N, O, S, NO, SO, SO₂, wherein the aforementioned ring is unsubstituted or substituted with one or more R^{a} or OR⁴;
each R⁷ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl,
phenyl, and a 3-, 4-, 5- or 6-membered saturated, partially unsaturated or aromatic heterocyclic ring containing 1, 2 or 3 heteroatoms or heteroatom groups selected from N, O, S, NO, SO, SO₂;
each R^{a} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆ alkyl, C₁-C₆ haloalkyl, C₂-C₆ alkenyl, C₂-C₆ haloalkenyl, each unsubstituted or substituted with one or two radicals selected from C₁-C₄ alkoxy and C₁-C₄ haloalkoxy,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from the group consisting of C₁-C₆-alkyl, C₁-C₆-alkoxy, C₁-C₆ haloalkoxy and (C₁-C₆-alkoxy)carbonyl;
each R^{b} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl;
each n is independently 1 or 2.

35. The method of claim 27, wherein the symbols in formula (I) have the following meanings:
R is R′, NR′₂, C(═O)R′, C(═O)OR′, or C(═O)NR′₂;
each R′ is independently hydrogen;
C₁-C₆-alkyl, C₂-C₆-alkenyl, each substituted with one or more R¹,
phenyl, substituted with one or two R²,
or is selected from the group consisting of: 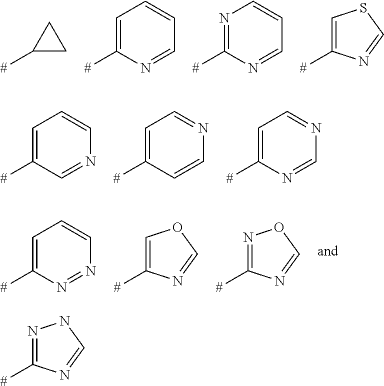
wherein each of the above ring systems is unsubstituted or substituted with one or more R²;
each R¹ is independently halogen;
each R² is independently halogen, C₁-C₆ alkyl, C₁-C₆ haloalkyl or OR⁴; and
each R⁴ is independently C₁-C₆ alkyl or C₁-C₆ haloalkyl.

36. The method of claim 27, wherein the symbols in formula (I) have the following meanings:
R is R′, NR′₂, C(═O)R′, C(═O)OR′ or C(═O)NR′₂;
each R′ is hydrogen,
C₁-C₆-alkyl, C₂-C₆-alkenyl, each substituted with one or more R¹,
phenyl, substituted with one or two R²,
or is selected from A1 to A28: 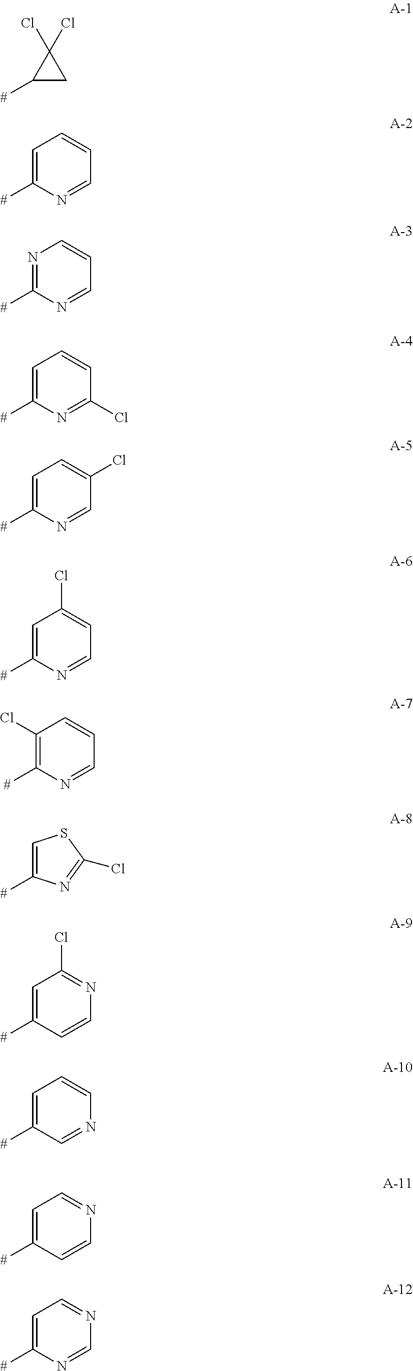 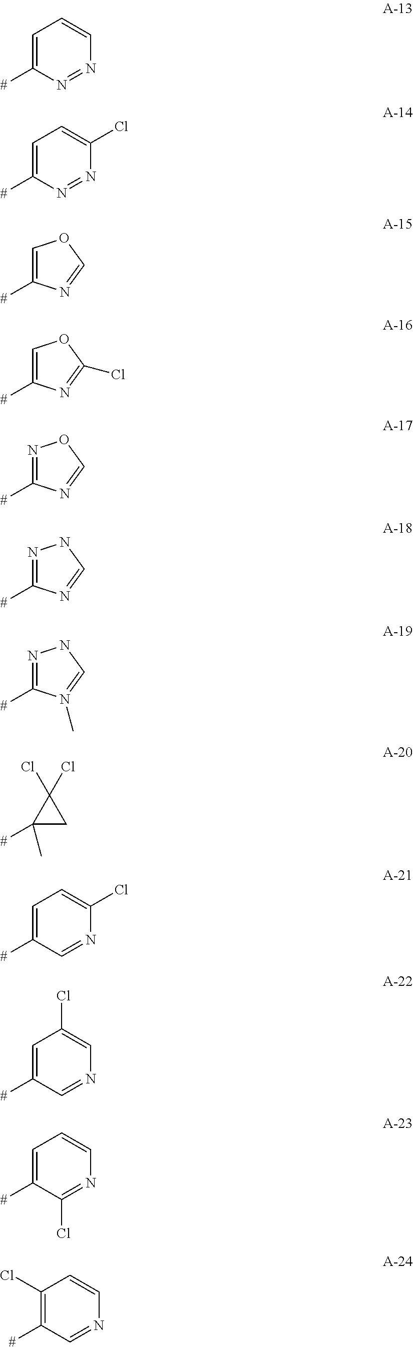 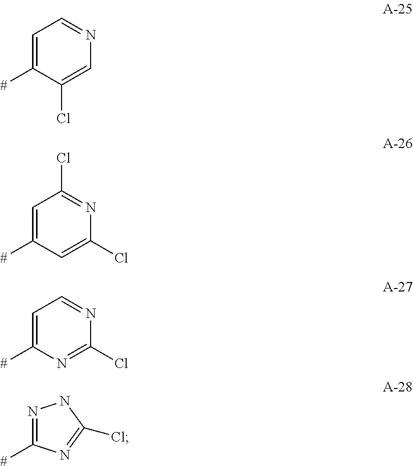
each R¹ is independently halogen;
each R² is independently halogen, C₁-C₆ alkyl, C₁-C₆ haloalkyl or OR⁴;
each R⁴ is independently C₁-C₆ alkyl or C₁-C₆ haloalkyl.

37. The method of claim 29, wherein
R is R′, C(═O)R′, C(═O)OR′, C(═O)NR′₂, C(═S)R′, C(═S)OR′, C(═S)NR′₂, C(═NR″)R′, C(═NR″)NR′₂, S(O)ₙR′, S(O)ₙNR′₂ or NR′₂;
each R′ is independently selected from the group consisting of hydrogen, C₁-C₆-alkyl, C₂-C₆-alkenyl, wherein the carbon atoms of the aforementioned aliphatic radicals are unsubstituted or substituted with one or more R¹, cyano,
C₃-C₈-cycloalkyl, C₅-C₆-cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R″ is independently selected from the group consisting of hydrogen,
C₁-C₆ alkyl, C₂-C₆ alkenyl, each unsubstituted or substituted with one or more R¹,
C₃-C₈ cycloalkyl, C₅-C₆ cycloalkenyl, each unsubstituted or substituted with one or more R²,
phenyl, unsubstituted or substituted with up to five R²,
and a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²;
each R′″ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl and phenyl;
each R¹ is independently halogen, cyano,
phenyl, unsubstituted or substituted with up to five R²,
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R²,
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, or NR⁵—CO—NR⁵₂;
each R² is independently halogen, cyano,
C₁-C₆-alkyl, C₂-C₆-alkenyl, wherein the carbon atoms of the aforementioned aliphatic radicals are unsubstituted or substituted with one or more R^{b},
C₃-C₈-cycloalkyl, C₅-C₆-cycloalkenyl, each unsubstituted or substituted with one or more R^{a},
phenyl, unsubstituted or substituted with up to five R^{a},
a 3-, 4-, 5- or 6-membered saturated, partly unsaturated or aromatic heterocyclic ring, comprising 1, 2 or 3 heteroatom units selected from the group consisting of O, S, S(═O), S(═O)₂, N and N(O), which heterocyclic ring is unsubstituted or substituted with one or more R^{a},
C═(O)R³, C(═O)OR⁴, C(═O)NR⁵₂, C(═S)R³, C(═S)OR⁴, C(═S)NR⁵₂, S(═O)ₙR⁴, S(═O)ₙNR⁵₂, OR⁴, SR⁴, NR⁵₂, OS(O)₂R⁴, NR⁵—S(O)₂—R⁴, NR⁵—S(O)₂—NR⁵₂, NR⁵—COOR⁴, NR⁵—CO—NR⁵₂, OS(O)₂R⁴, NR⁵—S(O)₂—R⁴, NR⁵—S(O)₂—NR⁵₂, or NR⁵—CO—OR⁴;
each R³, R⁴, R⁵ is independently hydrogen, C₁-C₆ alkyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from N, O, S, NO, SO, and SO₂, wherein the aforementioned ring is unsubstituted or substituted with one or more R^{a};
each R⁶ is independently hydrogen, C₁-C₆ alkyl, C₂-C₆ alkenyl, wherein the carbon atoms of the aforementioned aliphatic radicals are unsubstituted or substituted with one or more R^{b},
C₃-C₈ cycloalkyl or C₅-C₆ cycloalkenyl, wherein the carbon atoms of the aforementioned cycloaliphatic radicals are unsubstituted or substituted with one or more R^{a},
phenyl unsubstituted or substituted with up to 5 R^{a},
or a 3-, 4-, 5- or 6-membered saturated, partially unsaturated or aromatic heterocyclic ring containing 1, 2 or 3 heteroatoms or heteroatom groups selected from the group consisting of N, O, S, NO, SO, SO₂, wherein the aforementioned ring is unsubstituted or substituted with one or more R^{a} or OR⁴;
each R⁷ is independently selected from the group consisting of C₁-C₆ alkyl, C₁-C₆ haloalkyl, C₁-C₆ alkoxyalkyl, C₂-C₆ alkenyl, C₂-C₆ haloalkenyl, C₃-C₈ cycloalkyl, C₃-C₈ halocycloalkyl, C₁-C₆ haloalkoxyalkyl,
phenyl, and a 3-, 4-, 5- or 6-membered saturated, partially unsaturated or aromatic heterocyclic ring containing 1, 2 or 3 heteroatoms or heteroatom groups selected from N, O, S, NO, SO, SO₂;
each R^{a} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆ alkyl, C₁-C₆ haloalkyl, C₂-C₆ alkenyl, C₂-C₆ haloalkenyl, each unsubstituted or substituted with one or two radicals selected from C₁-C₄ alkoxy and C₁-C₄ haloalkoxy,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
each R^{b} is independently selected from the group consisting of halogen, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio,
C₃-C₈ cycloalkyl, C₅-C₆ cycloalkenyl, C₃-C₈ halocycloalkyl, C₅-C₆ halocycloalkenyl, each unsubstituted or substituted with one or two radicals selected from C₁-C₄ alkyl and C₁-C₄ alkoxy,
phenyl, benzyl, pyridyl, and phenoxy, wherein the four last mentioned radicals are unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl;
each n is independently 1 or 2.

38. The method of claim 29, wherein the symbols in formula (I) have the following meanings:
R is R′, NR′₂, C(═O)R′, C(═O)OR′, or C(═O)NR′₂;
each R′ is independently hydrogen;
C₁-C₆-alkyl, C₂-C₆-alkenyl, each substituted with one or more
phenyl, substituted with one or two R²,
or is selected from the group consisting of: 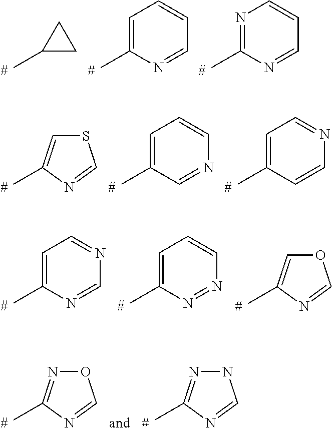
wherein each of the above ring systems is unsubstituted or substituted with one or more R²;
each R¹ is independently halogen;
each R² is independently halogen, C₁-C₆ alkyl, C₁-C₆ haloalkyl or OR⁴; and
each R⁴ is independently C₁-C₆ alkyl or C₁-C₆ haloalkyl.

39. The method of claim 29, wherein the symbols in formula (I) have the following meanings:
R is R′, NR′₂, C(═O)R′, C(═O)OR′ or C(═O)NR′₂;
each R′ is hydrogen,
C₁-C₆-alkyl, C₂-C₆-alkenyl, each substituted with one or more R¹,
phenyl, substituted with one or two R²,
or is selected from A1 to A28: 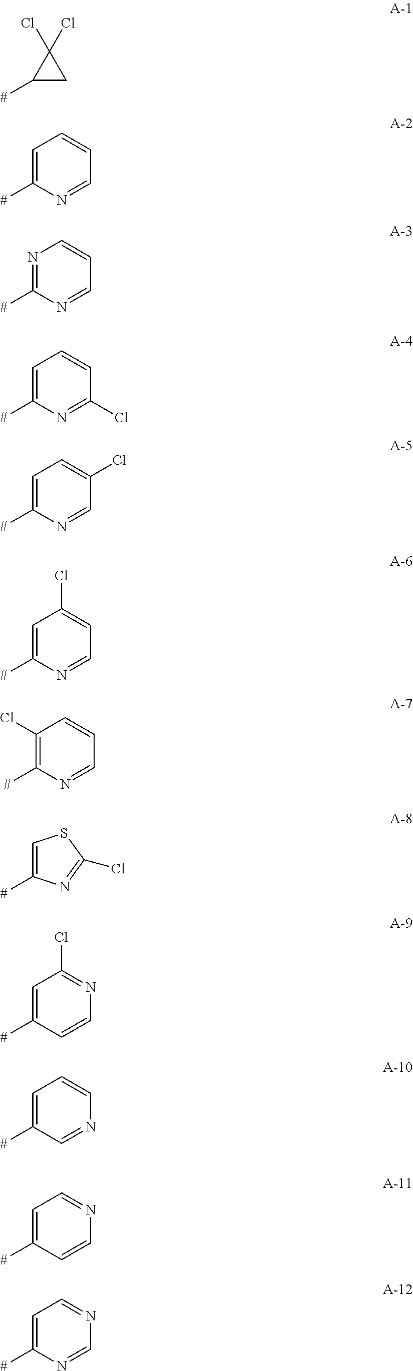 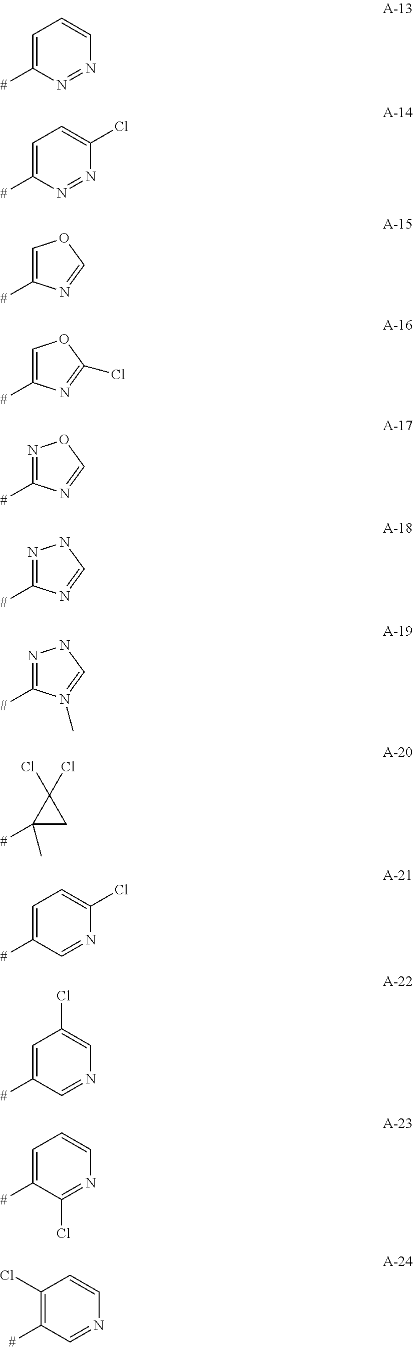 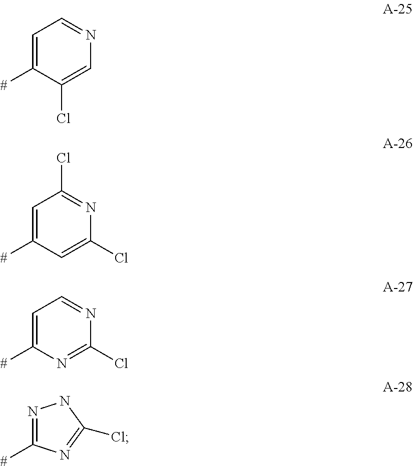
each R¹ is independently halogen;
each R² is independently halogen, C₁-C₆ alkyl, C₁-C₆ haloalkyl or OR⁴;
each R⁴ is independently C₁-C₆ alkyl or C₁-C₆ haloalkyl.
